SEQUENCE LISTING (2 sequences)

SEQ ID NO: 1 - protein, 466 aa, Homo sapiens
MSTRSVSSSSYRRMFGGPGTASRPSSSRSYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTFLIKTVETRDGQVINETSQHHDDLE

SEQ ID NO: 2 - protein, 438 aa, Homo sapiens
SYVTTSTRTYSLGSALRPSTSRSLYASSPGGVYATRSSAVRLRSSVPGVRLLQDSVDFSLADAINTEFKNTRTNEKVELQELNDRFANYIDKVRFLEQQNKILLAELEQLKGQGKSRLGDLYEEEMRELRRQVDQLTNDKARVEVERDNLAEDIMRLREKLQEEMLQREEAENTLQSFRQDVDNASLARLDLERKVESLQEEIAFLKKLHEEEIQELQAQIQEQHVQIDVDVSKPDLTAALRDVRQQYESVAAKNLQEAEEWYKSKFADLSEAANRNNDALRQAKQESTEYRRQVQSLTCEVDALKGTNESLERQMREMEENFAVEAANYQDTIGRLQDEIQNMKEEMARHLREYQDLLNVKMALDIEIATYRKLLEGEESRISLPLPNFSSLNLRETNLDSLPLVDTHSKRTFLIKTVETRDGQVINETSQHHDDLE

(19) United States
(12) Patent Application Publication
(10) Pub. No.: US 2007/0248539 A1
(43) Pub. Date: Oct. 25, 2007

(54) AgRM2 antigen

(75) Inventors: Mark C. Glassy, San Diego, CA (US); Michael E. McKnight, Poway, CA (US)

Correspondence Address: WILSON SONSINI GOODRICH & ROSATI, 650 PAGE MILL ROAD, PALO ALTO, CA, 94304-1050, US

(73) Assignee: Shantha West Inc., San Diego, CA (US)

(21) Appl. No.: 11/789,376

(22) Filed: Apr. 23, 2007

Related U.S. Application Data
(60) Provisional application No. 60/745,484, filed on Apr. 24, 2006.

(51) Int. Cl. A61K 51/00; G01N 33/574; C07H 21/04; C12P 21/06; A61K 39/395; C07K 14/82; C07K 16/30

(52) U.S. Cl. 424/1.49; 435/7.23; 435/69.1; 435/320.1; 435/325; 530/350; 530/388.8; 424/155.1; 536/23.5

(57) ABSTRACT

The antigen recognized by the antibody produced by a deposited cell line (ATCC accession no. PTA 5411) has been shown to be a fragment of vimentin. This vimentin fragment will be useful in therapy of and screening for cell proliferative disorders such as cancer.

### CROSS-REFERENCE

This application claims the benefit of U.S. provisional application Ser. No. 60/745,484 filed Apr. 24, 2006, which is incorporated by reference in its entirety.

### BACKGROUND OF THE INVENTION

Since cancers are heterogeneous, thus no single therapy may be as effective as a combination. A heterogeneous disease may require heterogeneous therapy to maximize response; this forms a theoretical basis for combination therapy protocols. See Glassy M C & McKnight M E, “Requirements for human antibody cocktails for oncology,” Expert Opin Biol Ther. 5: 1333-38 (2005); Glassy M C & Koda K, “The nature of an ideal therapeutic human antibody,” Expert Opin. Biol Ther. 2: 1-2 (2002)). Combination chemotherapy has generally been proven to be more effective than individual drugs in the treatment of cancer. This concept may now be applied to therapy using drugs produced by biotechnology employing combinations of recombinant molecules.

Decreasing tumor burden is a major goal of cancer therapy. Antibodies used individually as monotherapy have been shown to be effective in eliminating tumor cells, thus demonstrating their potential in the clinic. See Dillman R O, “Monoclonal antibodies in the treatment of malignancy: basic concepts and recent developments,” Cancer Invest. 19: 833-41 (2001); Blattman J N and Greenberg P D, “Cancer immunotherapy: a treatment for the masses,” Science 305: 200-205 (2004); Slamon D J et al., “Use of chemotherapy plus a monoclonal antibody against HER2 for metastatic breast cancer that over expresses HER2,” N. Eng. J. Med 344: 783-92 (2001); King K M & Younes A, “Rituximab: review and clinical applications focusing on non-Hodgkin's lymphoma,” Expert Rev. Anticancer Ther. 1: 177-186 (2001); Brekke O H & Sandlie I, “Therapeutic antibodies for human diseases at the dawn of the twenty-first century,” Nat Rev Drug Discov 2: 52-62 (2003); Milenic D E, “Monoclonal antibody-based therapy strategies: providing options for the cancer patient,” Curr Pharm Des 8: 1749-64 (2002); Koda K et al., “Immunotherapy for recurrent colorectal cancers with human monoclonal antibody, SK-1,” Anticancer Res. 21: 621-28 (2001).

Antibody therapy dates to 1890 (Behring E A von and Kitasato S, “Uber das zustandekomrnen der diphtherie immunitat und der tetanus immunitat bei thieren,” Dtsch. Med. Wochenschr 16: 1113-14 (1890)) and now monoclonal antibodies (Mabs) are considered among the most effective immunotherapy techniques and are routinely used in the clinic. See Dillman R O, “Monoclonal antibodies in the treatment of malignancy: basic concepts and recent developments,” Cancer Invest. 19: 833-841 (2001); Blattman J N and Greenberg P D, “Cancer immunotherapy: a treatment for the masses,” Science 305: 200-205 (2004); Slamon D J et al., “Use of chemotherapy plus a monoclonal antibody against HER2 for metastatic breast cancer that over expresses HER2,” N. Eng. J. Med 344: 783-792 (2001); King K M and Younes A, “Rituximab: review and clinical applications focusing on non-Hodgkin's lymphoma,” Expert Rev. Anticancer Ther. 1: 177-186 (2001); Brekke O H and Sandlie I, “Therapeutic antibodies for human diseases at the dawn of the twenty-first century,” Nat Rev Drug Discov 2: 52-62 (2003); Milenic D E, “Monoclonal antibody-based therapy strategies: providing options for the cancer patient,” Curr Pharm Des 8: 1749-64 (2002); Koda K, “Immunotherapy for recurrent colorectal cancers with human monoclonal antibody, SK-1,” Anticancer Res. 21: 621-28 (2001).

Studies using combinations of murine monoclonal antibodies have shown additive and even synergistic responses thus suggesting that cocktails of antibodies will be useful in the clinic. See Elliott E V et al., “Synergistic catatonic effects of antibodies directed against different cell surface determinants,” Immunology 34: 405-409 (1978); Hellstrom I. et al., “Monoclonal antibodies to two determinants of melanoma-antigen p97 act synergistically in complement-dependent cytotoxicity,” J. Immunol. 127: 157-60 (1981); Ziegler-Heitbrock H W L et al., “Protection of mice against tetanus toxin by combination of two human monoclonal antibodies recognizing distinct epitopes on the toxin molecule,” Hybridoma 5: 21-31 (1986). Similar results have been obtained with human Mabs. See Hagiwara H and Aotsuka Y, “Cytotoxicity of mixed human monoclonal antibodies reacting to tumor antigens,” Acta Paediatr. Jpn 29: 552-556 (1987); Glassy M C & Trass K G, “Use of a cocktail of human MAbs to probe tumor antigen distribution,” FASEB J. 2: A1836 (1988).

Additionally, cocktails of antibodies which show enhanced cell killing may be due to combined apoptosis mechanisms. See Glassy M C and McKnight M E, “Requirements for human antibody cocktails for oncology,” Expert Opin Biol Ther. 5:1333-38 (2005). A study by Pohle et al. showed that antibodies can enhance apoptosis when used as combinations. See Pohle T et al., “Lipoptosis: tumor-specific cell death by antibody-induced intracellular lipid accumulation,” Cancer Res 64: 3900-06 (2004). It is known that patients can mount an antibody response to their own tumor antigens. See Brandlein S et al., “Natural IgM antibodies and immunosurveillance mechanisms against epithelial cancer cells in humans,” Cancer Res. 63: 7995-8005 (2003); Stockert E et al., “A survey of the humoral immune response of cancer patients to a panel of human tumor antigens.” J Exp Med 187: 1349-1354 (1998); Vollmers H P & Brandlein S, “Nature's best weapons to fight cancer. Revival of human monoclonal IgM antibodies,” Human Antibod 11: 131-142 (2002); Glassy M C et al., “Lessons learned about the therapeutic potential of the natural human immune response to lung cancer,” Exp. Opin. Invest. Drugs 8: 995-1006 (1999); Kotlan B. et al., “Novel ganglioside antigen identified by B cells in human medullary breast carcinomas. The proof of principle concerning the tumor infiltrating B lymphocyte,” J. Immunol. 175: 2278-2285 (2005). Also various complex immunosurveillance mechanisms interact to generate the cellular and humoral responses. See Blattman J N & Greenberg P D, “Cancer immunotherapy: a treatment for the masses,” Science 305: 200-205 (2004); Burnet M, “Immunological surveillance in neoplasia,” Transplant Rev 7: 3-25 (1971); Pardoll D, “Does the immune system see tumors as foreign or self?,” Ann. Rev. Immunol. 21: 807-839 (2003); Shankaran V et al., “IFN-gamma and lymphocytes prevent primary tumour development and shape tumour immunogenicity,” Nature 410: 1107-1111 (2001); Dunn, G P et al., “The Three Es of Cancer Immunoediting,” Ann. Rev. Immunol. 22: 329-360 (2004).

The presence of “new antigen” or “neo-epitopes” from the tumor stimulates the immune response and germinal center development. See MacLennan I C, “Germinal centers,” Ann Rev Immunol 12: 117-139 (1994); Cody H S (ed), “Sentinal Lymph Node Biopsy,” (Martin Dunitz Ltd, London. (2002). The immune response can be oligoclonal, that is, the response occurs by multiple germinal centers in various immune organs, such as sentinal lymph nodes (Cody H S (ed): “Sentinal Lymph Node Biopsy,” (Martin Dunitz Ltd, London. 2002). The neo-epitopes may be altered genes or proteins and thus susceptible to recognition by the immune response and may potentially serve as molecular targets in oncology. In this way, the human immune response can serve as a drug discovery program to identify effective antigens which may then serve as the focus of targeted therapy. See Glassy M C and McKnight M E, “A novel drug discovery program utilizing the human immune response, Curr. Opin. Invest. Drugs 2: 853-858 (1993); Glassy M C and McKnight M E, “Pharming the human lymph node,” Exp. Opin. Invest. Drugs 3: 1057 (1994).

A series of human monoclonal antibodies have been generated from the immune response to antigens in a series of tumors. The inventors have developed a panel of mAbs derived from draining lymph nodes of cancer patients (RM1, RM2, RM3, and RM4). See Glassy M C and McKnight M E, “Requirements for human antibody cocktails for oncology,” Expert Opin Biol Ther. 5: 1333-38 (2005). The mAb RM1 was derived from NSCL cancer lymph nodes; the mAb RM2 was derived from colon/pancreatic lymph nodes; the mAb RM3 was derived from colon/pancreatic lymph nodes; and the mAb RM4 was derived from colon lymph nodes. The RM2 and RM4 antibodies are the subject of the pending U.S. patent application Ser. No. 10/662,044, which is hereby incorporated by reference in its entirety including any drawings, figures, and tables.

Glassy, McKnight, and coworkers have compared data obtained from cell lines with that of tissue specificity using the RM series of antibodies and demonstrated a close correlation. See Chang H R et al., “Tumor-associated antigens recognized by human monoclonal antibodies,” Ann. Surg. Oncol. 1: 213-21 (1994). The expression of antigen on the cells which bind RM antibody varies both quantitatively and qualitatively, both in the case of cell lines and tissue samples. Log-phase cells express more antigen than stationary phase cells. Immunohistological evaluation of the various tissue sections shows areas of intense, moderate and light staining suggesting that the tumor samples are heterogeneous.

In general, the cell line specificity profiles of the RM antibodies closely parallel those seen with tissue samples. However, in the case of RM2, although it was reactive with cell lines derived from pancreatic and ovarian tumors, it was poorly immunohistochemically reactive with tissues obtained directly from pancreatic and ovarian tumors. The specificity profiles as seen by the RM panel of Mabs suggest that only subsets of the cells being assayed express the recognized antigens. In the case of RM2, the decided contrast between the immunohistochemical results compared to cell line expression may be due, in part, to the tumor cells in situ being in various stages of cell cycle/cell during proliferation. Thus, there is a need to develop effective cancer therapies based on identification and utilization of the RM2 antigen.

### SUMMARY OF THE INVENTION

In some embodiments, the polypeptide's amino acid backbone's sequence comprises a fragment of an amino acid sequence set forth in SEQ ID NO:1. In some embodiments, the polypeptide's amino acid backbone's sequence comprises a carboxy-terminal fragment of the amino acid sequence set forth in SEQ ID NO:1. In some embodiments, the polypeptide's amino acid backbone's sequence comprises an amino acid sequence that lacks at least about 20 amino acids from the polypeptide's amino terminus relative to the sequence set forth in SEQ ID NO:1. In some embodiments, the polypeptide's amino acid backbone's sequence comprises an amino acid sequence that lacks between about 25 and about 28 amino acids from the polypeptide's amino terminus relative to the sequence set forth in SEQ ID NO:1. In some embodiments, the polypeptide's amino acid backbone's sequence comprises an amino acid sequence that lacks between about 25 and about 30 amino acids from the polypeptide's amino terminus relative to the sequence set forth in SEQ ID NO:1. In some embodiments, the polypeptide's amino acid backbone lacks between about 25 and about 35 amino acids from the polypeptide's amino terminus relative to the amino acid sequence set forth in SEQ ID NO:1. In some embodiments, the polypeptide's amino acid backbone's sequence comprises an amino acid sequence that lacks at least about 28 amino acids from the polypeptide's amino terminus relative to the sequence set forth in SEQ ID NO:1. In some embodiments, the polypeptide's amino acid backbone's sequence comprises an amino acid sequence that lacks at least about 30 amino acids from the polypeptide's amino terminus relative to the amino acid sequence set forth in SEQ ID NO:1. In some embodiments, the polypeptide's amino acid backbone's sequence comprises an amino acid sequence that lacks at least about 40 amino acids from the polypeptide's amino terminus relative to the sequence set forth in SEQ ID NO:1. In some embodiments, the polypeptide is less than about 440 amino acids in length. In some embodiments, the polypeptide is less than about 410 to about 440 amino acids in length. In some embodiments, the polypeptide is less than about 400 amino acids in length. In some embodiments, the polypeptide is isolated from a mammal. In some embodiments, the polypeptide is isolated from a human. In some embodiments, the polypeptide is a vimentin variant. In some embodiments, the vimentin variant comprises a fragment of an amino acid sequence selected from the group of amino acid sequences consisting of GenBank Accession Nos. AF058445; AF058446; AF044286; AF058444; NM138609; NM138609; BC013331; AF054174; AF041483; NM138610.1; and NM004893.2.

In another aspect, the instant invention relates to an isolated polypeptide comprising a fragment of a polypeptide having an amino acid sequence as set forth in SEQ ID NO:1 wherein the fragment is a carboxy-terminal fragment that specifically binds to a human monoclonal antibody produced by a cell line deposited as ATCC accession no. PTA 5411, and the fragment's amino acid sequence comprises less than about 440 amino acids of the amino acid sequence set forth in SEQ ID NO:1. In some embodiments, the fragment's amino acid sequence comprises less than about 420 amino acids of the amino acid sequence set forth in SEQ ID NO:1. In some embodiments, the fragment's amino acid sequence comprises less than about 400 amino acids of the amino acid sequence set forth in SEQ ID NO:1. In some embodiments, the fragment's amino acid sequence comprises less than about 350 amino acids of the amino acid sequence set forth in SEQ ID NO:1.

In certain aspects, the instant invention relates to a nucleic acid encoding the polypeptide of any one of the prior aspects and/or embodiments.

In other aspects, the instant invention relates to a transformed cell that contains the polypeptide of any one of the prior aspects and/or embodiments.

In further aspects, the instant invention relates to a vector comprising a nucleic acid encoding the polypeptide of any one of the prior aspects and/or embodiments.

In still other aspects, the instant invention relates to a transformed cell comprising a nucleic acid encoding the polypeptide of any one of the prior aspects and/or embodiments. In some embodiments, the transformed cell is prokaryotic; in some embodiments, the transformed cell is eukaryotic.

In another aspect the instant invention relates to a pharmaceutical composition comprising the polypeptide of any one of the prior aspects and/or embodiments, and a pharmaceutically acceptable carrier.

The instant invention also relates to a kit comprising the polypeptide of any one of the prior aspects and/or embodiments.

The instant invention also relates to a method of identifying a cell that expresses a polypeptide that specifically binds to a human monoclonal antibody produced by a cell line deposited as ATCC accession no. PTA 5411, comprising screening the cell for expression of the polypeptide. In some embodiments, the screening comprises detecting a nucleic acid that encodes the polypeptide. In some embodiments, the cell is present in a subject. In some embodiments, the subject is a mammal. In some embodiments, the subject is a human.

In another aspect, the instant invention relates to method of screening for a cell proliferative disorder comprising analyzing a biological sample for a polypeptide that specifically binds to a human monoclonal antibody produced by a cell line deposited as ATCC accession no. PTA 5411. In some embodiments, the screening comprises detecting the polypeptide's presence. In some embodiments, the screening comprises detecting a nucleic acid that encodes the polypeptide. In some embodiments, the screening is in vivo. In some embodiments, the cell proliferative disorder is a tumor or a benign hyperplasia. In some embodiments, the tumor is metastatic. In some embodiments, the tumor is a stage I, II, or III tumor, or the tumor is a stage IV or V tumor. In some embodiments, the tumor is a solid tumor. In some embodiments, the tumor is a T-cell lymphoma, and in some of these embodiments, may be Sezary Syndrome. In some embodiments, the cell proliferative disorder is derived from cells selected from the group consisting of breast, colon, gut, lung, brain, skin and pancreas cells.

In a further aspect, the instant invention relates to a method of inducing or increasing an immune response to a polypeptide that specifically binds to a human monoclonal antibody produced by a cell line deposited as ATCC accession no. PTA 5411 comprising administering to a subject a sufficient amount of the polypeptide to elicit the immune response to the polypeptide in the subject. In some embodiments, the immune response comprises a cell-mediated immune response. In some embodiments, the immune response comprises a humoral immune response.

In a still further aspect, the instant invention relates to a method of treating a cell proliferative disorder comprising administering to a subject a sufficient amount of a polypeptide that specifically binds to a human monoclonal antibody produced by a cell line deposited as ATCC accession no. PTA 5411 to treat the cell proliferative disorder. In some embodiments, the cell proliferative disorder is a tumor.

In another aspect, the instant invention relates to a method of treating a subject having, or at risk of having, a tumor comprising administering to the subject a sufficient amount of a polypeptide that specifically binds to a human monoclonal antibody produced by a cell line deposited as ATCC accession no. PTA 5411 to treat the subject. In some embodiments, the subject has a tumor. In some embodiments, the tumor comprises a stage IV or V tumor. In some embodiments, the tumor comprises cells derived from cells selected from the group consisting of breast, colon, gut, lung, brain, skin or pancreas. In some embodiments, the tumor is a T-cell lymphoma, and in some of these embodiments, may be Sezary Syndrome. In some embodiments, the treatment reduces tumor volume, inhibits an increase in tumor volume, inhibits progression or metastasis of the tumor, stimulates tumor cell apoptosis, or reduces tumor metastasis. In some embodiments, the treatment reduces one or more adverse symptoms associated with the tumor. In some embodiments, the method further comprises administering an additional anti-tumor or immune system-enhancing agent or treatment. In some embodiments, the additional anti-tumor or immune system-enhancing agent is an antibody, a radioisotope, a toxic agent, an immunotherapeutic agent, a chemotherapeutic agent or external radiation. In some embodiments, the chemotherapeutic agent comprises an agent selected from the group consisting of an alkylating agent, an anti-metabolite, a plant extract, a plant alkaloid, nitrosourea, a hormone, a nucleoside and a nucleotide analogue. In some embodiments, the chemotherapeutic agent comprises an agent selected from the group consisting of cyclophosphamide, colchicine, colcemid, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC), bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin and dibromomannitol.

In a further aspect, the instant invention relates to a method of identifying an inhibitor or stimulator of the expression of a polypeptide that specifically binds to a human monoclonal antibody produced by a cell line deposited as ATCC accession no. PTA 5411 comprising: (a) contacting a cell that is capable of expressing the polypeptide that specifically binds to a human monoclonal antibody produced by the cell line with a test compound; and (b) detecting expression of the polypeptide, wherein a change the expression of the polypeptide indicates that the test compound is an inhibitor or stimulator of the expression of the polypeptide. In some embodiments, the contacting is in vivo. In some embodiments, the contacting is in vitro.

In yet another aspect, the instant invention relates to a method of screening a subject having or at risk of having a cell proliferative disorder, wherein the cell proliferative disorder is derived from a tissue selected from breast, colon, gut, lung, brain, skin, pancreas and the lymphatic system comprising the steps of: analyzing the subject for expression of a polypeptide that specifically binds to a human monoclonal antibody produced by a cell line deposited as ATCC accession no. PTA 5411, wherein the polypeptide's presence in the tissue identifies the subject as having or at risk of having a cell proliferative disorder.

In the above aspects and embodiments, the instant invention relates to fragments and subsequences of vimentin. In the aspects of the invention that relates to a composition including vimentin, such a composition may comprise, consist of, or consist essentially of fragments and subsequences of vimentin.

### BRIEF DESCRIPTION OF THE DRAWINGS

FIG. 1 depicts the amino acid sequence of vimentin as well as the AgRM2 fragment (underlined). Also shown is the sequence, both DNA and amino acid, in the vicinity of the cleavage of the vimentin backbone to form the AgRM2 fragment. AgRM2 (i.e., a truncated vimentin protein) is composed of 438 amino acids with a molecular weight of approximately from 50 to 52 kDa (calculated to be 50.74 kDa). The DNA sequence below begins with TCC encoding Serine at position 29.

### DETAILED DESCRIPTION

In the instant invention, the antigen associated with a human monoclonal antibody RM2 has been identified and characterized. This antigen, AgRM2, has been discovered to be a fragment of the intermediate filament protein, vimentin.

Vimentin is normally a protein found in the intermediate filaments of many, but not all, cells. See Herrmann H and Aebi U, “Intermediate filaments: molecular structure, assembly mechanism, and integration into functionally distinct intracellular scaffolds,” Ann. Rev. Biochem. 73: 49-89 (2004). Vimentin has been used as a histological marker in cancer diagnosis. For example, recently it was retroactively correlated with a poor prognosis in kidney cancer. See Moch et al., “High-throughput tissue microarray analysis to evaluate genes uncovered by cDNA microarray screening in renal cell carcinoma,” Am J Pathol. 154(4): 981-86 (1999). The vimentin fragment recognized by RM 2 is surprisingly found located on the cell surface, at least in part, in contrast to its normal intracellular location.

The biodistribution data demonstrates tumor localization confirming the ability of the RM Mabs to locate antigen-positive cells in vivo. The biodistribution data together with the xenograft data strongly suggests that the RM panel of human antibodies can be utilized in vivo with clinical protocols and that a clinical immunotargeting program is feasible.

The instant invention provides, in part, the isolation and characterization of AgRM2, an antigen associated with cell proliferative disorders including, but not limited to, metastatic and non-metastatic tumors. The invention also provides antibodies directed to AgRM2 and their use in therapy, diagnosis, or prognosis of cell proliferative disorders including, but not limited to, metastatic and non-metastatic tumors.

AgRM2 is initially identified as a fragment, or subsequence, of the protein vimentin with a mobility in denaturing gel electrophoresis of 52 kDa. In contrast to vimentin, AgRM2 is expressed, at least in part, on the cell surface. AgRM2 is more highly expressed in proliferating cells than in non-proliferating cells, thus possibly explaining the discrepancy noted above between cell line data and immunohistochemistry. Furthermore, AgRM2 expression is associated with tumors including, but not limited to, those arising from the breast cancer, the lung cancer, and the pancreas cancer, as well as melanoma. AgRM2 is therefore useful for diagnosis, prognosis and as a target for the treatment of cell proliferative disorders.

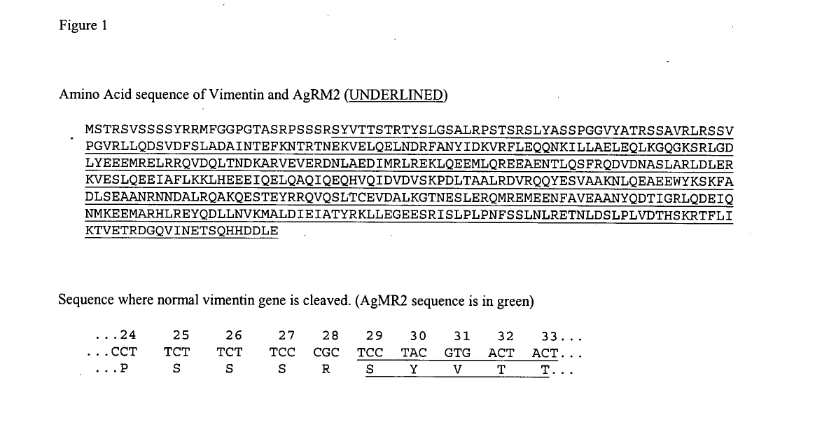

Without being bound to a particular theory, it is believed that the cleavage shown in FIG. 1 triggers a series of unusual protein folding events for the AgRM2 peptide as compared to the native full-length vimentin peptide. In some embodiments, various AgRM2 sequences of the invention do not react to commercially available anti-vimentin antibody. In some embodiments, various folded AgRM2 sequences of the invention create different epitopes for antibody recognition as compared to the native full-length vimentin peptide.

In some embodiments, the AgRM2 sequences of the invention comprise the amino acid sequence as shown in SEQ ID NO:2 with the proviso that the native full-length vimentin peptide sequences are specifically excluded. In some embodiments, the AgRM2 sequences of the invention comprise various deletions of the amino acid sequence as shown in SEQ ID NO:2 with the proviso that the native full-length vimentin peptide sequences are specifically excluded. For example, the AgRM2 sequences of the invention may have a deletion at the amino terminus of SEQ ID NO:2 with at least 1, 2, 3, 4, 5, 6, 7, 8, 9, 10, 11-15, 16-20, or 21-50 amino acids deleted.

The instant invention provides isolated and purified fragments of vimentin, and nucleic acids that encode fragments of vimentin. In some embodiments, an isolated or purified vimentin fragment specifically binds to a human monoclonal antibody produced by the cell line deposited as ATCC accession no. PTA 5411. In various aspects, the vimentin fragment has a deletion at the amino terminus relative to full length native human vimentin (e.g., of at least 10, 20, 25, 26, 27, 28, 29, 30, 31, 32, 33, 34, 45, 40, 45, 50, 51, 55, 60, 70, 80 amino acids from the amino terminus relative to full length native human vimentin). In various additional aspects, the vimentin polypeptide sequence consists of a polypeptide having no more than 50 to 100, 100 to 200, 200 to 300, 300 to 400, 400 to 410, 410 to 420, 420 to 430, 430 to 435, 436, 437, 438, 439, 440, 440 to 444, 444 to 450, or 450 to 460 amino acids in length. Vimentin polypeptide sequences include mammalian vimentin sequences, including human (as set forth in FIG. 1; see, e.g., Genbank Accession Nos. P08670 and M14144), polymorphic variants thereof (for example, polypeptides encoded by a sequence selected from Genbank Accession Nos. BAD96227, Q548L2, AK222482, and AK222507 or selected from Genbank Accession Nos. AF058445; AF058446; AF044286; AF058444; NM138609; NM138609; BC013331; AF054174; AF041483; NM138610.1; and NM004893.2), and other variants.

Huet and co-workers have also recently observed in malignant Sezary cells a correlation with the expression of a fragment of vimentin with the malignant state of the cells. See Huet D et al., “SC5 mAb Represents a Unique Tool for the Detection of Extracellular Vimentin as a Specific Marker of Sézary Cells,” The Journal of Immunology 176: 652-659 (2006). It is noted that an atypical cell-surface location of vimentin can be associated with malignancy. Huet and co-workers, however, associated their antibody binding epitope with the 1B region of vimentin in the amino-terminal half of vimentin.

Furthermore, secretion of vimentin has been observed in activated monocyte-derived macrophages See Mor-Vaknin N et al., “Vimentin is secreted by activated macrophages,” Nat Cell Biol. 5(1): 59-63 (2003). Also, other workers have potentially identified vimentin as a CTCL-associated antigen. See Hartmann T B et al., “SEREX identification of new tumour-associated antigens in cutaneous T-cell lymphoma,” Br J Dermatol. 150(2): 252-58 (2004).

Schietinger and colleagues describe that a somatic mutation in the chaperone gene Cosmc abolished function of a glycosyltransferase, disrupting O-glycan Core 1 synthesis and creating a tumor-specific glycopeptidic neo-epitope consisting of a monosaccharide and a specific wild-type protein sequence. See Schietinger et al., “A mutant chaperone converts a wild-type protein into a tumor-specific antigen”, Science 314: 304-308 (2006). Accordingly, there appears a reasonably common event in cancer biology which AgRM2 also belongs where normal cellular proteins are processed differently by various mechanisms to generate tumor-specific antigens.

The terms “protein,” “polypeptide,” and “peptide” are used herein interchangeably and are used in their conventional meaning, i.e., a chain of amino acids. None of the terms imply any set length or range of lengths of amino acid chains. These terms also do not imply or exclude post-translational (or post-synthetic) modifications of the polypeptide, e.g., glycosylations, acetylations, phosphorylations, and the like, as well as other modifications known to those of skill in the art, both naturally occurring and non-naturally occurring. A polypeptide may be an entire protein, or a region of the amino acid chain. A protein may comprise more than one polypeptide.

In some embodiments, a polypeptide chain or backbone is formed by a series of amino acids joined by peptide bonds and/or amide bonds. The amino acids may also be linked by non-natural and non-amide chemical bonds including, for example, those formed with glutaraldehyde, N-hydroxysuccinimide esters, bifunctional maleimides, or N,N′-dicyclohexylcarbodiimide (DCC). Non-amide bonds include, for example, ketonmethylene, aminomethylene, olefin, ether, thioether and the like. See Spatola, “Peptide and Backbone Modifications,” in Chemistry and Biochemistry of Amino Acids, Peptides and Proteins, Vol. 7, pp 267-357, (Marcel Decker, NY; 1983).

As used herein, the term “isolated,” when used as in reference to a compound of the instant invention (e.g., a vimentin fragment of the instant invention, a nucleic acid encoding a vimentin fragment of the instant invention, and modified forms of a vimentin fragment of the instant invention, a fragment of a vimentin fragment of the instant invention, cells containing and/or expressing a vimentin fragment of the instant invention, and vectors encoding a vimentin fragment of the instant invention), means that such a compound is prepared by human activity or is separated from its natural state; i.e., that, if it occurs in nature, it has been removed from its original environment by human activity.

Generally, an “isolated” compound is substantially free of one or more materials with which it normally associates with in nature; for example, other proteins, nucleic acids, lipids, carbohydrates, and/or cell membranes have been separated from the composition of the instant invention. Thus, an isolated protein is typically substantially free of one or more materials with which it may typically associate with in nature. The term “isolated”does not exclude alternative physical forms, such as polypeptide multimers, post-translational modifications (e.g., phosphorylation, glycosylation) or derivatized forms.

Thus, following isolation, a polypeptide compound of the instant invention may be joined to other polypeptides to form fusion proteins, either during synthesis or post-synthesis (e.g., translation), and/or may be joined to other compounds, such as a dye. The isolated polypeptides can be found in host cells, both in culture or in whole organisms. Introduced into host cells in culture or in whole organisms, such polypeptides still would be isolated, as the term is used herein, because they would not be in their naturally occurring form or environment. Similarly, polypeptides may occur in a composition, such as a media formulations, solutions for introduction of polypeptides into, for example, cells, compositions or solutions for chemical or enzymatic reactions or antibody binding, for instance, which are not naturally occurring compositions, and, therein remain isolated polypeptides within the meaning of that term as it is employed herein.

An “isolated” protein may also be “purified” when free of most or all of the materials with which it typically associates with in nature. For example, the term “purified” in reference to a polypeptide does not require absolute purity (such as a homogeneous preparation); instead, it represents an indication that the sequence is relatively purer than in the natural environment. Compared to the natural level this level should be at least 2- to 5-fold greater (e.g., in terms of mg/mL). Purification of at least one order of magnitude, in some embodiments two or three orders, and in some embodiments four or five orders of magnitude is expressly contemplated. In some embodiments, the substance is free of contamination at a functionally significant level, for example 90%, 95%, or 99% pure. Purity may be determined by any appropriate method, including, for example, UV spectroscopy, chromatography (e.g., HPLC, gas phase), gel electrophoresis (e.g., silver or Coomassie staining) and sequence analysis (nucleic acid and peptide). Of course, a “purified” molecule may be combined with one or more other molecules. Thus, the term “purified” does not exclude combination compositions made by the hand of man.

In reference to a nucleic acid, the term “isolating” refers to removing a naturally occurring nucleic acid molecule of a given sequence from its normal cellular environment. Thus, the nucleic acid molecule may be in a cell-free solution or placed in a different cellular environment. The term does not imply that the nucleic acid molecule is the only nucleotide chain present, but that it is essentially free (about 90-95% pure at least) of non-nucleotide material naturally associated with it, and thus is distinguished from isolated chromosomes. Also, by the use of the term “isolating” in reference to nucleic acid is meant that the specific DNA or RNA molecule is increased to a significantly higher fraction (2- to 5-fold) of the total DNA or RNA present in the solution of interest than in the cells from which the sequence was taken. In some embodiments, this could be caused by a person reducing the amount of other DNA or RNA present, or by increasing in the amount of the specific DNA or RNA molecule, or by a combination of the two. However, it should be noted that enriched does not imply that there are no other DNA or RNA molecules present, just that the relative amount of the nucleic acid molecule of interest has been significantly increased.

The term “significant” is used to indicate that the level of increase is useful to the person making such an increase, and generally means an increase relative to other nucleic acids of at least about 2-fold, in some embodiments at least about 5- to about 10-fold or even more. The DNA from other sources may, for example, comprise DNA from a yeast or bacterial genome, or a cloning vector such as pUC19. This term distinguishes from naturally occurring events, such as viral infection, or tumor-type growths, in which the level of one mRNA may be naturally increased relative to other species of mRNA. That is, the term is meant to cover only those situations in which a person has intervened to elevate the proportion of the desired nucleic acid. The term “isolating” may also include a step wherein an RNA sequence is converted into a cDNA sequence by techniques well known in the art, or that is synthesized as the sense or complementary antisense strand.

Isolated DNA sequences are relatively more pure than in the natural environment (compared to the natural level this level should be at least 2- to 5-fold greater, e.g., in terms of mg/mL). Individual sequences obtained from PCR may be purified to electrophoretic homogeneity. The DNA molecules obtained from this PCR reaction could be obtained from total DNA or from total RNA. These DNA sequences are not necessarily naturally occurring, but rather are in some embodiments obtained via manipulation of a partially purified naturally occurring substance (e.g., mRNA). For example, the construction of a cDNA library from mRNA involves the creation of a synthetic substance (cDNA) and pure individual cDNA clones can be isolated from the synthetic library by clonal selection from the cells carrying the cDNA library. The process which includes the construction of a cDNA library from mRNA and isolation of distinct cDNA clones yields an approximately about 10⁶-fold purification of the native message. Thus, purification of at least one order of magnitude, in some embodiments two or three orders, and in some embodiments four or five orders of magnitude is expressly contemplated.

The term “antibody” refers to a protein that binds to other molecules (antigens) via heavy and light chain variable domains, V_{H} and V_{L}, respectively. Antibodies include, but are not limited to, IgG, IgD, IgA, IgM and IgE, subtypes, and mixtures thereof. Antibodies may be polyclonal or monoclonal, intact immunoglobulin molecules, two full length heavy chains linked by disulfide bonds to two full length light chains, or fragments (i.e. fragments) thereof, with our without constant region, that bind to an epitope of an antigen, and mixtures thereof. Antibodies may comprise heavy or light chain variable regions, V_{H} or V_{L} individually, or in any combination.

Polypeptides and nucleic acids of the invention include modified or variant forms. The term “modify” and grammatical variations thereof, when used in reference to a composition such as a polypeptide or nucleic acid, means that the modified composition deviates from a reference composition. Polypeptide modifications include amino acid substitutions, additions and deletions (also known as subsequences and fragments); such polypeptide modifications are optionally referred to as “variants.” Polypeptide modifications also include one or more D-amino acids substituted for L-amino acids (and mixtures thereof), structural and functional analogues, for example, peptidomimetics having synthetic or non-natural amino acids or amino acid analogues and derivative forms.

Polypeptide modifications further include fusion (chimeric) polypeptides, in other words a polypeptide with an amino acid sequence having one or more amino acids present in the polypeptide chain not normally present in a reference native (wild type) polypeptide covalently attached to the native (wild type) polypeptide molecule, for example, one or more amino acids of non-vimentin attached to a vimentin polypeptide sequence that specifically binds to a human monoclonal antibody produced by the cell line deposited as ATCC accession no. PTA 5411. Such chimeric polypeptides may comprise polypeptide fragments derived from apoptotic factors, differentiation factors, toxins, chemokines and cytokines (interleukins, interferons).

By the term “specifically binding,” as used herein, is meant that an antibody, or other molecule, binds to another molecule, i.e., the target, with greater affinity than two unrelated molecules bind under the specified conditions. Antibodies or antibody fragments are polypeptides that contain regions that can specifically bind other polypeptides. In some embodiments of the instant invention, “specifically binds” means that binding between the two molecules has an about 10⁴-fold greater affinity, an about 10⁵-fold greater affinity, an about 10⁶-fold greater affinity, an about 10⁷-fold greater affinity, an about 10⁸-fold greater affinity, an about 10⁹-fold greater affinity than the two molecules bind to unrelated molecules.

Modifications may include cyclic structures such as an end-to-end amide bond between the amino and carboxy-terminus of the polypeptide molecule or intra- or inter-molecular disulfide bond. Polypeptides may be modified in vitro or in vivo, e.g., post-translationally, modified to include, for example, sugar residues, phosphate groups, ubiquitin, fatty acids or lipids.

Modifications should do not eliminate an activity or function of a given reference composition (e.g., binding to a human monoclonal antibody produced by the cell line deposited as ATCC accession no. PTA 5411). A modified protein may have an amino acid substitution, addition or deletion (e.g., 1-3, 3-5, 5-10 or more amino acids). In some embodiments, the substitution is a conservative amino acid substitution.

A “conservative amino acid substitution” means the replacement of one amino acid in the polypeptide chain by a biologically, chemically or structurally similar residue. “Biologically similar” means that the substitution is compatible with the biological activity of the protein being modified, e.g., the modified polypeptide still binds to a human monoclonal antibody produced by the cell line deposited as ATCC accession no. PTA 5411. “Structurally similar” means that the amino acids have side chains with similar length, such as interchanging alanine with serine. “Chemical similarity” means that the residues have the same charge or are both hydrophilic or hydrophobic. Particular examples include the substitution of one hydrophobic residue, such as isoleucine, valine, leucine or methionine for another, or the substitution of one polar residue for another, such as the substitution of arginine for lysine, glutamic for aspartic acids, or glutamine for asparagine, serine for threonine, and the like.

Modified polypeptides include those with amino acid sequences having 50%, 60%, 70%, 75%, 80%, 85%, 90%, 95%, 96%, 97%, 98%, or more identity to a sequence of vimentin and/or AgRM2, as set forth in FIG. 1. The identity can be over any defined region or fragment of the protein.

The term “identity” and grammatical variations thereof, mean that two or more referenced sequences and/or entities are the same. Thus, where two protein sequences are identical, they have the same amino acid sequence. “Areas of identity” means that a portion of two or more referenced entities are the same in a given region. Thus, where two protein sequences are identical over one or more sequence regions they share identity in these regions. The term “substantial identity” means that the identity is structurally or functionally significant. That is, the identity is such that the molecules are structurally identical or have at least one of the same functions (e.g., biological function) even though the molecules may be somewhat different. Due to variation in the amount of sequence conservation between structurally and functionally related proteins, the amount of sequence identity for substantial identity will depend upon the type of protein, the region involved, and any function. There may be as little as 30% sequence identity for proteins to have substantial identity, but typically there is more, e.g., 50%, 60%, 75%, 85%, 90%, 95%, 96%, 97%, 98%, identity to a reference sequence. For nucleic acid sequences, 50% sequence identity, or more, typically constitutes substantial homology, but again may vary depending on the comparison region and its function, if any.

The extent of identity between two sequences can be ascertained using computer programs and/or mathematical algorithms known in the art. Such algorithms that calculate percent sequence identity (homology) generally account for sequence gaps and mismatches over the comparison region. For example, a BLAST (e.g., BLAST 2.0) search algorithm (see Altschul et al., J. Mol. Biol. 215: 403-10 (1990)) has exemplary search parameters as follows: Mismatch −2; gap open 5; gap extension 2. The default parameters may also be used. For polypeptide sequence comparisons, a BLASTP algorithm is typically used in combination with a scoring matrix, such as PAM100, PAM 250, and BLOSUM 62.

As used herein, the term “subsequence” or “fragment” in reference to a nucleic acid molecule, nucleic acid sequence, protein molecule and/or amino acid sequence means a portion of a given full length polypeptide relative to the known length and amino acid sequence of a peptide. For example, vimentin is generally accepted to be 466 amino acids in length. Thus, a fragment of vimentin is at least one amino acid less in length than full length vimentin (e.g., one or more terminal amino acid deletions from either amino or carboxy termini, and/or one or more internal deletions). A fragment of vimentin wherein the amino terminal serine is deleted is said to lack one amino acid from the amino terminus. Fragments may be any length up to almost the length of the full length reference molecule. Fragments may have deletions from the amino terminus relative to the full length protein as set forth in a GenBank listing of about 10, about 20, about 25, about 26, about 27, about 28, about 29, about 30, about 31, about 32, about 33, about 34, about 45, about 40, about 45, about 50, about 51, about 55, about 60, about 70, about 80 amino acids. In various additional aspects, the fragment of vimentin may consist of a polypeptide comprising no more than 50 to 100, 100 to 200, 200 to 300, 300 to 400, 400 to 410, 410 to 420, 420 to 430, 430 to 435, 436, 437, 438, 439, 440, 440 to 444, 444 to 450, or 450 to 460 amino acids of the full length vimentin sequence. The term “fragment” also may be similarly used in reference to an amino acid sequence or a nucleic acid sequence where the term means that a portion of the sequence has been deleted relative to the full length sequence but that the sequence is otherwise intact.

Fragments include portions which retain at least part of the function or activity of the reference full length polypeptide. Fragments may also acquire a function or activity absent from the reference full length polypeptide. For example, a protein fragment may display an epitope that is absent from or hidden in a full length sequence. In some embodiments, an isolated or purified vimentin fragment of the instant invention binds to a human monoclonal antibody produced by the cell line deposited as ATCC accession no. PTA 5411, whereas full-length wild type vimentin does not exhibit detectable specific binding to the monoclonal antibody produced by the cell line deposited as ATCC accession no. PTA 5411.

As used herein, the term “carboxy-terminal fragment” is used in reference to a polypeptide that has a specific parent sequence of amino acids and the term refers to a fragment of the polypeptide that is reduced in length with respect to the full-length specific parent sequence by deletion of amino acids from the amino-terminal end while leaving the sequence of amino acids leading up to and including the carboxy-terminal amino acid intact. In some embodiments, the specific sequence of amino acids is SEQ ID NO:1, a carboxy-terminal fragment of vimentin.

In some embodiments, the specific sequence of amino acids is SEQ ID NO:2. The term “amino-terminal fragment” as used herein has a similar meaning, but the deletions are made from the carboxy-terminal end and leaves the amino-terminal sequence intact. As used herein, neither carboxy-terminal fragment nor amino-terminal fragment is meant to imply that the fragment is synthesized in a longer form and then the polypeptide chain is shortened; the carboxy-terminal fragment or amino-terminal fragment may be synthesized in vivo or in vitro in an already truncated form with respect to the specific parent sequence of amino acids.

Modified polypeptides and nucleic acids may include one or more non-native (wild-type) functions, or be “multifunctional,” which means that the composition referred to has one or more different or additional activities or functions not found in vimentin or in a vimentin fragment as found in vivo. Particular non-limiting examples include, for example, enzyme activity, ligand or receptor binding (substrates, agonists and antagonists), detection, purification, and toxicity.

A particular example of an additional function is a “detectable label,” which refers to a molecule that enables detection of the conjugated molecule. Examples of detectable labels include chelators, photoactive agents, radioisotopes and radionuclides (alpha, beta and/or gamma emitters), fluorescent agents and paramagnetic ions. Radioactive labels include, for example, ³H, ¹⁴C, ³²P, ³³P, ³⁵S, ¹²⁵I, and ¹³¹I. Non-radioactive labels include moieties such as gold particles, colored glass or plastic polystyrene, polypropylene, or latex beads, and amino acid sequences such as tags, as set forth herein. Detectable labels include those that display enzyme activity (e.g., green fluorescent protein, acetyltransferase, galactosidase, glucose oxidase, peroxidase, horseradish peroxidase (HRP), urease, luciferase and alkaline phosphatase). Detectable fluorescent compounds include, for example, fluorescein isothiocyanate, rhodamine, phycoerytherin, phycocyanin, allophycocyanin, o-phthaldehyde, fluorescamine, and commercially available fluorophores such as Alexa Fluor 350, Alexa Fluor 488, Alexa Fluor 532, Alexa Fluor 546, Alexa Fluor 568, Alexa Fluor 594, Alexa Fluor 647, and BODIPY dyes such as BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY TR, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine and Texas Red).

Other detectable compounds include, for example, colloidal metals, chemiluminescent compounds (e.g., luminol, isoluminol, an aromatic acridinium ester, an imidazole, an acridinium salt and oxalate esters), bioluminescent compounds (e.g., luciferin, luciferase and aequorin), paramagnetic labels (e.g., chromium (III), manganese (II), manganese (III), iron (II), iron (III), cobalt (II), nickel (II), copper (II), praseodymium (III), neodymium (III), samarium (III), gadolinium (III), terbium (III), dysprosium (III), holmium (III), erbium (III) and ytterbium (III)) which can be detected by MRI, and adhesion proteins (e.g., biotin, streptavidin, avidin, and other lectins).

Another particular example of an additional function is a “tag,” which refers to a molecule conjugated to another that allows detection, iolation or purification. Specific examples of tags include immunoglobulins, T7, polyhistidine tags, glutathione-S-transferase, a chitin-binding tag, calmodulin-binding tag, myc tag, and a Xpress epitope (detectable by anti-Xpress™ antibody; Invitrogen, Carlsbad, Calif., USA).

Additional candidate forms of chimeric polypeptides include those that display cytotoxicity (e.g., bacterial cholera toxin, pertussis toxin, anthrax toxin lethal factor, Pseudomonas exotoxin A, diphtheria toxin, plant toxin ricin, cytotoxic drugs and radionuclides such as ⁴⁷Sc, ⁶⁷Cu, ⁷²Se, ⁸⁸Y, ⁹⁰Sr, ⁹⁰Y, ⁹⁷Ru, ⁹⁹Tc, ¹⁰⁵Rh, ¹¹¹In, ¹²⁵I, ¹³¹I, ¹⁴⁹Tb, ¹⁵³Sm, ¹⁸⁶Re, ¹⁸⁸Re, ¹⁹⁴Os, ²⁰³Pb, ²¹¹At, ²¹²Bi, ²¹³Bi, ²¹²Pb, ²²³Ra, ²²⁵Ac, ²²⁷Ac, ²²⁸Th. Modified polypeptides and nucleic acids therefore also include addition of functional entities, molecules, or other moieties, covalently or non-covalently attached to the polypeptides and nucleic acids of the invention.

Polypeptides, including modified forms such as substitutions, additions, and deletions (e.g., subsequences and fragments), may be produced using recombinant technology with nucleic acids encoding a polypeptide via expression in cells or using in vitro translation. Polypeptide sequences, including antibodies, may also be produced by a chemical synthesizer (see, e.g., Applied Biosystems, Foster City, Calif.); and polypeptides thus produced may include modified and/or chimeric polypeptides. Antibodies may be expressed from recombinantly produced antibody-encoding nucleic acid. See Harlow and Lane, Using Antibodies: A Laboratory Manual (Cold Spring Harbor Laboratory, 1999); Fitzgerald et al., J.A.C.S. 117: 11075 (1995); Gram et al., Proc. Natl. Acad. Sci. USA 89:3576 (1992). In some embodiments, enzymes such as proteases (e.g., pepsin and papain) can be used to generate subsequences and fragments following polypeptide synthesis.

The invention also provides nucleic acids encoding and vectors containing nucleic acids encoding invention polypeptides, including fragments and/or modified forms of vimentin. Also provided are transformed cells that contain vimentin fragments or modified vimentin fragments of the instant invention. Transformed cells including nucleic acids encoding vimentin fragment of the invention are further provided. Transformed cells include prokaryotic and eukaryotic cells, such as bacteria, fungi, insect and mammalian cells in culture, ex vivo or in vivo.

As used herein, “nucleic acid,” refers to at least two or more ribonucleic acid or deoxyribonucleic acid base pairs (nucleotides) linked through a phosphoester bond or equivalent. Nucleic acids include polynucleotides and polynucleosides. Nucleic acids include circular and linear, single, double and triplex molecules. A nucleic acid molecule may belong exclusively to or be in a mixture of, but not limited to: RNA, DNA, cDNA, genomic nucleic acids, non-genomic nucleic acids, naturally occurring nucleic acids, non-naturally occurring nucleic acids and synthetic nucleic acids.

Nucleic acids may be of any length, and typically range from about 20 nucleotides to 10 Kb, 10 nucleotides to 5 Kb, 1 to 5 Kb or less, 1000 to about 500 nucleotides or less in length. Nucleic acids may also be shorter, for example, 100 to about 500 nucleotides, or from about 12 to 25, 25 to 50, 50 to 100, 100 to 250, or about 250 to 500 nucleotides in length.

Nucleic acids further include modifications such as nucleotide and nucleoside substitutions, additions and deletions, as well as derivatized forms and fusion sequences (e.g., encoding chimeric polypeptides containing a vimentin fragment). For example, due to the degeneracy of the genetic code, nucleic acids include sequences and fragments degenerate with respect to nucleic acids that encode the amino acid sequence of vimentin as set forth in FIG. 1. Other examples are nucleic acids complementary to a sequence that encodes vimentin as set forth in FIG. 1. Nucleic acids encoding vimentin fragments have from about 10 to 25, 25 to 50, 50 to 100, 100 to 200, 200 to 300, 300 to 500, 500 to 1000 nucleotides, or more. Such nucleic acids are useful for expressing vimentin polypeptides or fragments thereof, for genetic manipulation (as primers and templates for PCR amplification), and as probes to detect the presence or an amount of a nucleic acid molecule encoding vimentin or a vimentin fragment of the instant invention in vitro, in a cell, culture medium, biological sample (e.g., tissue, organ, blood or serum), or in a subject in vivo.

The terms “substantial sequence homology” or “substantially similar” as used herein in reference to nucleic acid sequences may refer to two nucleic acid sequences having minor and non-consequential sequence variations from each other. In this regard, the “minor and non-consequential” sequence variations mean that the substantially similar sequences will function in substantially the same manner to produce substantially the same compositions as the nucleic acid compositions referenced herein. In some embodiments, such substantially similar sequences have 80% or more nucleic acid identity, in some embodiments 90% or more nucleic acid identity, or in some embodiments 95% or more nucleic acid identity.

Nucleic acid identity is a property of nucleic acid sequences that measures their similarity or relationship. Identity is measured by dividing the number of identical bases in the two sequences by the total number of bases and multiplying the product by 100. Thus, two copies of exactly the same sequence have 100% identity, while sequences that are less highly conserved and have deletions, additions, or replacements have a lower degree of identity. Those of ordinary skill in the art will recognize that several computer programs are available for performing sequence comparisons and determining sequence identity. A nucleic acid sequence that is substantially similar to a claimed sequence of the instant invention means that such a sequence may be within the scope of the claims.

Nucleic acids that hybridize at high stringency to nucleic acids that encode a vimentin polypeptide of the instant invention, a fragment thereof and nucleic acid sequences complementary to the encoding nucleic acids, are provided. Hybridizing nucleic acids are useful for detecting the presence or an amount of a sequence encoding a vimentin fragment of the instant invention in vitro, or in a cell, culture medium, biological sample (e.g., tissue, organ, blood or serum), or in a subject in vivo.

The term “hybridize” refers to the binding between nucleic acid sequences. Hybridizing sequences will generally have more than about 50% homology, more than about 50% homology, more than about 60% homology, more than about 70% homology, more than about 80% homology, or more than about 90% homology to a nucleic acid that encodes the amino acid sequence of AgRM2, vimentin, or another vimentin fragment. The hybridization region between hybridizing sequences may extend over at least about 10-15 nucleotides, 15-20 nucleotides, 20-30 nucleotides, 30-50 nucleotides, 50-100 nucleotides, or about 100-200 nucleotides or more.

As is understood by one skilled in the art, the T_{M} (melting temperature) is the temperature at which binding between two nucleic acid sequences is no longer stable. For two sequences to bind, the temperature of a hybridization reaction must be less than the calculated T_{M} for the sequences under the hybridization conditions. The T_{M} is influenced by the amount of sequence complementarity, length, composition (% GC), type of nucleic acid (RNA vs. DNA), and the amount of salt, detergent and other components in the reaction (e.g., formamide). All of these factors are considered in establishing appropriate hybridization conditions. See the hybridization techniques and formula for calculating T_{M} described in Sambrook et al., In: Molecular Cloning: A Laboratory Manual, 3rd ed. (Cold Spring Harbor Laboratory Press, 2001).

Typically, wash conditions are adjusted to attain the desired degree of hybridization stringency. Thus, hybridization stringency can be determined empirically, for example, by washing under particular conditions, e.g., at low stringency conditions or high stringency conditions. Optimal conditions for selective hybridization will vary depending on the particular hybridization reaction involved. An example of high stringency hybridization conditions are as follows: 2×SSC/0.1% SDS at about 37° C. or 42° C. (hybridization conditions); 0.5×SSC/0.1% SDS at about room temperature (low stringency wash); 0.5×SSC/0.1% SDS at about 42° C. (moderate stringency wash); and 0.1×SSC/0.1% SDS at about 65° C. (high stringency wash).

Nucleic acids may be produced using various standard cloning and chemical synthesis techniques separately or in combination. Such cloning techniques include nucleic acid amplification, e.g., polymerase chain reaction (PCR), with genomic DNA or cDNA targets using primers (e.g., a degenerate primer mixture) capable of annealing to the AgRM2 encoding sequence; and chemical synthesis of nucleic acid sequences. The sequences produced may then be translated in vitro, or cloned into a plasmid, then propagated and/or expressed in a cell (e.g., microorganisms, such as yeast or bacteria; eukaryotes such as animals or mammalian cells; or in plants).

The invention further provides nucleic acid expression cassettes including a nucleic acid encoding a vimentin fragment of the instant invention operatively linked to an expression control element. The term “operatively linked” refers to a physical or functional relationship between the elements referred to that permit them to operate in their intended fashion. Thus, an expression control element, e.g., a promoter, “operatively linked” to a nucleic acid means that the control element modulates nucleic acid transcription and, as appropriate, translation of the transcript.

Physical linkage is not required for the elements to be operatively linked. For example, a minimal expression control element may be linked to a nucleic acid encoding a vimentin fragment of the instant invention. A second element that controls expression of an operatively linked nucleic acid encoding a protein that functions “in trans” can bind to the above minimal element to influence expression of the vimentin fragment. Because the second element regulates expression of the vimentin fragment, the second element is operatively linked to the nucleic acid encoding the vimentin fragment even though they are not physically linked.

The term “expression control element” refers to a nucleic acid region that influences expression of an operatively linked nucleic acid as known in the art. Promoters and enhancers are particular non-limiting examples of expression control elements. A “promotor” is a regulatory region of a nucleic acid chain capable of initiating transcription coding sequence that is downstream (in a 3′ direction). The promoter sequence includes nucleotides for facilitating transcription initiation and is, in general, proximate to the 5′ end of the coding region. Enhancers also regulate gene expression, but can function at a distance from the transcription start site of the gene to which it is operatively linked (e.g., at either 5′ or 3′ ends of the gene, as well as within the gene). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, binding sites for small regulatory RNA molecules, polyadenylation signal to provide proper polyadenylation of the transcript of a gene of interest, and properly positioned stop codons.

Expression control elements include “constitutive” elements such that transcription of the operatively linked nucleic acid occurs without a signal or stimuli. Expression control elements that confer expression in response to a signal or stimuli, which either increases or decreases expression of the operatively linked nucleic acid, are “regulatable.” A regulatable element that increases expression of the operatively linked nucleic acid in response to a signal or stimuli is referred to as an “inducible element.” A regulatable element that decreases expression of the operatively linked nucleic acid in response to a signal or stimuli is referred to as a “repressible element” (i.e., the signal decreases expression; when the signal is removed or absent, expression is increased).

Expression control elements include elements active in a particular tissue or cell type, and are referred to as “tissue-specific” expression control elements. Tissue-specific expression control elements are typically active in specific cell or tissue types because they are recognized by transcriptional activator proteins, or other regulators of transcription, that are unique to the specific cell or tissue type.

Expression control elements include full-length nucleic acid sequences, such as native promoter and enhancer elements, as well as fragments or nucleotide variants thereof (e.g., substituted/mutated or other forms that differ from native sequences) which retain all or part of full-length or non-variant control element function (confer regulation, e.g., retain some amount of inducibility in response to a signal or stimuli).

A variety of heterologous systems are available for functional expression of recombinant proteins and are well known to those skilled in the art. Such systems include bacteria (Strosberg, et al., Trends in Pharmacological Sciences 13: 95-98 (1992)), yeast (Pausch, Trends in Biotechnology 15: 487-494 (1997)), several kinds of insect cells (Vanden Broeck, Int. Rev. Cytology 164: 189-268 (1996)), amphibian cells (Jayawickreme et al., Current Opinion in Biotechnology 8: 629-634 (1997)) and several mammalian cell lines (CHO, HEK293, COS, etc.; see Gerhardt, et al., Eur. J. Pharmacology 334: 1-23 (1997)). These examples do not preclude the use of other possible cell expression systems, including cell lines obtained from nematodes. Additional information is described in international PCT Patent Application Publication WO 98/37177, which is incorporated by reference in its entirety.

For bacterial expression, constitutive promoters include T7, as well as inducible promoters such as pL of bacteriophage λ, plac, ptrp, ptac (ptrp lac hybrid promoter). In insect cell systems, constitutive or inducible promoters (e.g., ecdysone) may be used. In yeast, constitutive promoters include, for example, ADH or LEU2 and inducible promoters such as GAL (see, e.g., Ausubel et al., In: Current Protocols in Molecular Biology, Vol. 2, Ch. 13, ed., (Greene Publish. Assoc. & Wiley Interscience, 1988); Grant et al., In: Methods in Enzymology, 153:516-544, eds. Wu & Grossman (Acad. Press, N.Y., 1987); Glover, DNA Cloning, Vol. II, Ch. 3, (IRL Press, Wash., D.C., 1986); Bitter, In: Methods in Enzymology, 152: 673-684, eds. Berger & Kimmel, (Acad. Press, N.Y., 1987); Strathern et al., The Molecular Biology of the Yeast Saccharomyces Vols. I and II (Cold Spring Harbor Press, 1982).

For mammalian, or other eukaryotic, expression, constitutive promoters of viral, or other, origin may be used, including mammalian, eukaryotic, yeast, insect, phage, or bacterial promoters. Possible promoters include SV40 promoters, viral long terminal repeats (LTRs) and the like, or inducible promoters derived from the genome of mammalian cells (e.g., metallothionein IIA promoter; heat shock promoter, steroid/thyroid hormone/retinoic acid response elements) or from mammalian viruses (e.g., the adenovirus late promoter; the inducible mouse mammary tumor virus LTR) are used. Other constitutive promoters from eukaryotes or prokaryotes may also be used.

The invention also provides stably and transiently transformed cells and progeny thereof into which a nucleic acid molecule encoding a vimentin fragment of the instant invention has been introduced by means of recombinant DNA techniques in vitro, ex vivo or in vivo. The transformed cells may be propagated and the introduced nucleic acid transcribed, or encoded protein expressed. Transformed cells include but are not limited to prokaryotic and eukaryotic cells such as bacteria, fungi, plant, insect, and animal (e.g., mammalian, including human) cells. The cells may be present in culture, in a cell, tissue or organ ex vivo or a subject. A progeny cell may not be identical to the parental cell, since there may be mutations that occur during parental cell replication.

The term “transformed,” “transfected,” or “transduced” refers to different ways to incorporate nucleic acid (e.g., a transgene) or protein exogenous into the cell. Thus, “transformed cells” or “transfected cells” include cells into which, or a progeny of which, a nucleic acid or polypeptide has been introduced by means of recombinant DNA techniques. Cell transformation/transfection to produce such cells may be carried out as described herein or using techniques known in the art. Accordingly, methods of producing cells including the nucleic acids and polypeptides of the invention are also provided.

Typically, cell transformation with a nucleic acid employs a “vector,” a term that refers to a plasmid, a virus (i.e., a viral vector), or other vehicle known in the art that may be manipulated by insertion or incorporation of a nucleic acid sequence. For genetic manipulation “cloning vectors” may be employed, and to transcribe or translate the inserted polynucleotide “expression vectors” may be employed. Such vectors are useful for introducing nucleic acids, including a nucleic acid that encodes a vimentin fragment operatively linked with an expression control element, and expressing the vimentin fragment in vitro (e.g., in solution or in solid phase), in cells or in a subject in vivo.

A vector generally contains an origin of replication for propagation in a cell. Control elements, including expression control elements as set forth herein, present within a vector, may be included to facilitate transcription and translation.

Vectors may include a selection marker, i.e., a gene that allows for the selection of cells containing the gene. “Positive selection” refers to a process whereby only cells that contain the selection marker will be selected. Drug resistance is one example of a positive selection marker, and cells containing the marker will survive in culture medium containing the drug used for the selection, and cells lacking the marker will die. Selection markers include drug resistance genes such as neo, which confers resistance to G418; hyg^{r}, which confers resistance to hygromycin; puro which confers resistance to puromycin; and mtx which confers resistance to methotrexate. Other positive selection marker genes include genes that allow identification or screening of cells containing the marker. These genes include genes for fluorescent proteins (GFP and similar proteins), luciferase, the lacZ gene, the alkaline phosphatase gene, and surface markers such as CD8, among others. “Negative selection” refers to a process whereby cells containing a negative selection marker are not selected, for example, due to exposure to an appropriate negative selection agent. For example, cells which contain the herpes simplex virus-thymidine kinase (HSV-tk) gene (see Wigler et al., Cell 11: 223 (1977)) are sensitive to the drug gancyclovir. Similarly, the gpt gene renders cells sensitive to 6-thioxanthine.

Viral vectors included are those based on retroviral, adeno-associated virus (AAV), adenovirus, reovirus, lentivirus, rotavirus genomes, simian virus 40 (SV40) or bovine papilloma virus. See Cone et al., Proc. Natl. Acad. Sci. USA 81: 6349 (1984); Gluzman ed., Eukaryotic Viral Vectors, (Cold Spring Harbor Laboratory, 1982); Sarver et al., Mol. Cell. Biol. 1: 486 (1981)). Additional viral vectors useful for expression include parvovirus, rotavirus, Norwalk virus, coronaviruses, paramyxo and rhabdoviruses, togavirus (e.g., sindbis virus and semliki forest virus) and vesicular stomatitis virus (VSV).

Mammalian expression vectors include those designed for in vivo and ex vivo expression, such as AAV. See U.S. Pat. No. 5,604,090, which is incorporated by reference in its entirety. AAV vectors have previously been shown to provide expression of Factor IX in humans and in mice at levels sufficient for therapeutic benefit. See Kay et al., Nat. Genet. 24: 257 (2000); Nakai et al., Blood 91: 4600 (1998). Adenoviral vectors (described in U.S. Pat. Nos. 5,700,470, 5,731,172 and 5,928,944, which are all incorporated by reference in their entireties), herpes simplex virus vectors (see U.S. Pat. No. 5,501,979, which is incorporated by reference in its entirety) retroviral (e.g., lentivirus vectors are useful for infecting dividing as well as non-dividing cells and foamy viruses) vectors (see U.S. Pat. Nos. 5,624,820, 5,693,508, 5,665,577, 6,013,516 and 5,674,703 and international PCT Patent Application Publications WO92/05266 and WO92/14829, which are all incorporated by reference in their entireties) and papilloma virus vectors (e.g., human and bovine papilloma virus) have all been employed in gene therapy (see U.S. Pat. No. 5,719,054, which is incorporated by reference in its entirety). Vectors also include cytomegalovirus (CMV) based vectors (see U.S. Pat. No. 5,561,063, which is incorporated by reference in its entirety). Vectors that efficiently deliver genes to cells of the intestinal tract have been developed. See U.S. Pat. Nos. 5,821,235, 5,786,340 and 6,110,456, which are all incorporated by reference in its entireties.

Introduction of polypeptides and nucleic acids into target cells may also be carried out by methods known in the art such as osmotic shock (e.g., calcium phosphate), electroporation, microinjection, cell fusion, etc. Introduction of polypeptides and nucleic acids in vitro, ex vivo and in vivo may also be accomplished using other techniques. For example, a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. Polypeptides and nucleic acids may be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid drug delivery system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells, including polypeptides and nucleic acids, are known to one skilled in the art and include, for example, phosphatidylcholine, phosphatidylserine, lipofectin and DOTAP (Invitrogen, Carlsbad, Calif., USA). See U.S. Pat. Nos. 4,844,904, 5,000,959, 4,863,740, and 4,975,282, which are all incorporated by reference in its entireties. Piperazine based amphilic cationic lipids useful for gene therapy also are known. See U.S. Pat. No. 5,861,397, which is incorporated by reference in its entirety. Cationic lipid systems also are known. See U.S. Pat. No. 5,459,127, which is incorporated by reference in its entirety. In some embodiments, viral or non-viral vector means of delivery into cells or tissue, in vitro, in vivo or ex vivo are used.

A vimentin fragment of the instant invention and nucleic acids encoding such a polypeptide may be combined with any other compound or agent that provides an enhanced or synergistic therapeutic benefit. The invention therefore also provides combination compositions including a vimentin fragment of the instant invention or a nucleic acid encoding a vimentin fragment of the instant invention and one or more additional compounds or agents and methods of using the combinations. For example, a vimentin fragment of the instant invention may be combined with a compound or agent that has anti-cell proliferative (e.g., anti-tumor) activity or immune system-enhancing activity.

As used here, the term “anti-cell proliferative activity,” when used in reference to a compound, agent, therapy or treatment, means that the compound, agent, therapy or treatment, reduces or inhibits cell proliferation or growth, stimulates or promotes cell apoptosis, lysis, necrosis or differentiation. The term “immune system-enhancing,” when used in reference to a compound, agent, therapy or treatment, means that the compound, agent, therapy or treatment, provides an increase, stimulation, induction or promotion of an immune response, humoral or cell-mediated. Such anti-cell proliferative and immune system-enhancing therapies and treatments can reduce or inhibit cell proliferation or enhance immune response generally, or reduce or inhibit cell proliferation of, or enhance immune response towards, a specific target, such as a cell proliferative disorder (e.g., a tumor).

Specific non-limiting examples of anti-cell proliferative and immune system-enhancing agents include monoclonal, polyclonal antibody and mixtures thereof. Antibodies include antibodies that bind to tumor-associated antigens (TAA). A “tumor associated antigen” or “TAA” refers to an antigen expressed by a tumor cell. TAAs may be expressed in amounts greater in tumor cells than a normal non-tumor cell counterpart, or may be expressed at similar levels, or at levels less than a normal cell counterpart.

Particular non-limiting examples of TAAs that may be targeted and TAA binding antibodies include, for example, human IBD12 monoclonal antibody which binds to epithelial cell surface H antigen (see U.S. Pat. No. 4,814,275, which is incorporated by reference in its entirety); M195 antibody which binds to leukemia cell CD33 antigen (see U.S. Pat. No. 6,599,505, which is incorporated by reference in its entirety); monoclonal antibody DS6 which binds to ovarian carcinoma CA6 tumor-associated antigen (see U.S. Pat. No. 6,596,503, which is incorporated by reference in its entirety); and BR96 antibody which binds to Lex carbohydrate epitope expressed by colon, breast, ovary, and lung carcinomas. Additional antibodies having anti-tumor activity that may be employed include, for example, rituximab (Rituxan®), trastuzumab (Herceptin (anti-Her-2 neu antibody)), bevacizumab (Avastin), ibritumomab tiuxetan (Zevalin), tositumomab (Bexxar), Oncolym, 17-1A (Edrecolomab), 3F8 (anti-neuroblastoma antibody), MDX-CTLA4, alemtuzumab (Campathg), gemtuzumab (Mylotarg) cetuximab (Erbitux), edrecolomab (Panorex), and IMC-C225 (Cetuximab).

Other non-limiting examples of TAAs that may be targeted include MUC-1, HER-2/neu, MAGE, p53, T/Tn and CEA (breast cancer); MUC-2 and MUC-4, CEA, p53 and the MAGE (colon cancer); MAGE, MART-1 and gp100 (melanoma); GM2, Tn, sTn, Thompson-Friedenreich antigen (TF), MUC1, MUC2, beta chain of chorionic gonadotropin (hCG beta), HER2/neu, PSMA and PSA (prostate cancer); chorionic gonadotropin (testicular cancer); and alpha fetoprotein (hepato-cellular carcinoma).

Additional examples of immune system-enhancing agents include immune cells such as lymphocytes, plasma cells, macrophages, NK cells and B-cells expressing antibody against the tumor. Cytokines that enhance or stimulate immunogenicity against tumor such as IL-2, IL-1α, IL-1β, IL-3, IL-6; IL-7, granulocyte-macrophage-colony stimulating factor (GMCSF), IFN-γ, IL-12, TNF-alpha, and TNFbeta are also non-limiting examples of immune system-enhancing agents. Chemokines including MIP-1α, MIP-1β, RANTES, SDF-1, MCP-1, MCP-2, MCP-3, MCP-4, eotaxin, eotaxin-2, I-309/TCA3, ATAC, HCC-1, HCC-2, HCC-3, LARC/MIP-3α, PARC, TARC, CKβ, CKβ6, CKβ7, CKβ8, CKβ9, CKβ11, CKβ12, C10, IL-8, GROα, GROβ, ENA-78, GCP-2, PBP/CTAPIIIβ-TG/NAP-2, Mig, PBSF/SDF-1, and lymphotactin are additional non-limiting examples of immune system-enhancing agents.

An “anti-tumor,” “anti-cancer” or “anti-neoplastic” treatment, therapy, activity or effect refers to any compound, agent, therapy or treatment regimen or protocol that inhibits, decreases, slows, reduces or prevents hyperplastic, tumor, cancer or neoplastic growth, metastasis, proliferation or survival. Anti-tumor compounds, agents, therapies or treatments can operate by disrupting, inhibiting or delaying cell cycle progression or cell proliferation; stimulating or enhancing apoptosis, lysis or cell death or necrosis; stimulating or enhancing cell differentiation; inhibiting nucleic acid or protein synthesis or metabolism; and inhibiting cell division, or decreasing, reducing or inhibiting cell survival, or production or utilization of a necessary cell survival factor, growth factor or signaling pathway (extracellular or intracellular). Examples of anti-tumor therapy include chemotherapy, immunotherapy, radiotherapy (ionizing or chemical), local thermal (hyperthermia) therapy and surgical resection.

Specific non-limiting examples of agent classes having anti-cell proliferative and anti-tumor activities include alkylating agents, anti-metabolites, plant extracts, plant alkaloids, nitrosoureas, hormones, nucleoside and nucleotide analogues. Specific examples of drugs having anti-cell proliferative and anti-tumor activities include cyclophosphamide, colchicine, colcemid, azathioprine, cyclosporin A, prednisolone, melphalan, chlorambucil, mechlorethamine, busulphan, methotrexate, 6-mercaptopurine, thioguanine, 5-fluorouracil, cytosine arabinoside, AZT, 5-azacytidine (5-AZC) and 5-azacytidine related compounds, bleomycin, actinomycin D, mithramycin, mitomycin C, carmustine, lomustine, semustine, streptozotocin, hydroxyurea, cisplatin, mitotane, procarbazine, dacarbazine, taxol, vinblastine, vincristine, doxorubicin and dibromomannitol.

The invention further provides kits including one or more vimentin fragments of the instant invention and/or a nucleic acid encoding such a vimentin fragment of the instant invention, including pharmaceutical formulations, packaged into suitable packaging material. In one embodiment, a kit includes a vimentin fragment.

As used herein, the term “packaging material” refers to a physical structure housing the components of the kit. The packaging material may maintain the components sterilely, and may be made of material commonly used for such purposes (e.g., paper, corrugated fiber, glass, plastic, foil, ampules, etc.). The label or packaging insert may include appropriate written instructions, for example, practicing a method of the invention, e.g., diagnosing a cell-proliferative disorder, detecting a cell-proliferative disorder, treating a cell-proliferative disorder, etc. Kits of the invention therefore may additionally include instructions for using the kit components in a method.

Thus, in some embodiments, a kit includes a label or packaging insert including instructions for expressing a vimentin fragment of the instant invention or a nucleic acid encoding such an invention vimentin fragment in cells in vitro, in vivo, or ex vivo. In some embodiments, a kit includes a label or packaging insert including instructions for treating a subject (e.g., a subject having or at risk of having a cell proliferative disorder such as a tumor) with a vimentin fragment of the instant invention or a nucleic acid encoding a vimentin fragment of the instant invention in vivo, or ex vivo. In some embodiments, a kit includes a label or packaging insert including instructions for detecting the presence of, or the expression level of, AgRM2 in vitro or in vivo (e.g., to indicate or diagnose, or to provide a prognosis for, a subject having or at risk of having a cell proliferative disorder).

Instructions may therefore include instructions for practicing any of the methods of the invention described herein. For example, invention pharmaceutical compositions may be included in a container, pack, or dispenser together with instructions for administration to a subject. Instructions may additionally include indications of a satisfactory clinical endpoint or any adverse symptoms that may occur, or additional information required by regulatory agencies such as the Food and Drug Administration for use on a human subject.

The instructions may be on “printed matter,” e.g., on paper or cardboard within the kit, on a label affixed to the kit or packaging material, or attached to a vial or tube containing a component of the kit. Instructions may comprise voice or video tape and additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Kits of the instant invention may additionally include a buffering agent, a preservative, or a protein/nucleic acid stabilizing agent. The kit may also include control components for assaying for activity, e.g., a control sample or a standard. Each component of the kit may be enclosed within an individual container or in a mixture and all of the various containers may be within single or multiple packages.

Vimentin fragments of the instant invention and nucleic acids encoding vimentin fragments of the instant invention, including modified forms for vimentin fragments, may be incorporated into pharmaceutical compositions. Such pharmaceutical compositions are useful for administration to a subject in vivo or ex vivo, and for providing therapy for a physiological disorder or condition treatable with an invention vimentin polypeptide or nucleic acid, e.g., a cell-proliferative disorder (tumor) of the breast, colon, gut, brain, lung, skin or pancreas.

Pharmaceutical compositions include “pharmaceutically acceptable” and “physiologically acceptable” carriers, diluents or excipients. As used herein the terms “pharmaceutically acceptable” and “physiologically acceptable” include solvents (aqueous or non aqueous), solutions, emulsions, dispersion media, coatings, isotonic and absorption promoting or delaying agents, compatible with pharmaceutical administration. Such formulations may be contained in a liquid; emulsion, suspension, syrup or elixir, or solid form; tablet (coated or uncoated), capsule (hard or soft), powder, granule, crystal, or microbead. Supplementary compounds (e.g., preservatives, antibacterial, antiviral and antifungal agents) may also be incorporated into the compositions.

Pharmaceutical compositions may be formulated to be compatible with a particular local or systemic route of administration. Thus, pharmaceutical compositions include carriers, diluents, or excipients suitable for administration by particular routes. Specific non-limiting examples of routes of administration for compositions of the invention are parenteral, e.g., intravenous, intradermal, intramuscular, subcutaneous, oral, transdermal (topical), transmucosal, and rectal administration.

Solutions or suspensions used for parenteral, intradermal, or subcutaneous application may include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH of the composition may be adjusted with acids or bases, such as hydrochloric acid or sodium hydroxide.

Pharmaceutical compositions for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier may be a solvent or dispersion medium containing, for example, water, ethanol, polyol (for example, glycerol, propylene glycol, and liquid polyetheylene glycol, and the like), and suitable mixtures thereof. Fluidity may be maintained, for example, by the use of a coating such as lecithin, by the maintenance of the required particle size in the case of dispersion and by the use of surfactants. Antibacterial and antifungal agents include, for example, parabens, chlorobutanol, phenol, ascorbic acid and thimerosal. Isotonic agents, for example, sugars, polyalcohols such as mannitol, sorbitol, sodium chloride may be included in the composition. Including an agent which delays absorption, for example, aluminum monostearate and gelatin may prolong absorption of injectable compositions.

Sterile injectable solutions may be prepared by incorporating the active compound(s) in the required amount in an appropriate solvent with one or a combination of above ingredients followed by filtered sterilization. Generally, dispersions are prepared by incorporating the active compound into a sterile vehicle containing a basic dispersion medium and other ingredients as above. In the case of sterile powders for the preparation of sterile injectable solutions, methods of preparation include, for example, vacuum drying and freeze-drying which yields a powder of the active ingredient plus any additional desired ingredient from a previously sterile-filtered solution thereof.

For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays, inhalation devices (e.g., aspirators) or suppositories. For transdermal administration, the active compounds are formulated into ointments, salves, gels, creams or patches.

A vimentin fragment of the instant invention and nucleic acids encoding a vimentin fragment of the instant invention, including modified forms, may be prepared with carriers that protect against rapid elimination from the body, such as a controlled release formulation or a time delay material such as glyceryl monostearate or glyceryl stearate. The compositions may also be delivered using implants and microencapsulated delivery systems to achieve local or systemic sustained delivery or controlled release.

Biodegradable, biocompatable polymers may be used, such as ethylene vinyl acetate, polyanhydrides, polyglycolic acid, collagen, polyorthoesters, and polylactic acid. Methods for preparation of such formulations are known to those skilled in the art. The materials may also be obtained commercially from Alza Corporation and Nova Pharmaceuticals, Inc. Liposomal suspensions (including liposomes targeted to cells or tissues using antibodies or viral coat proteins) may also be used as pharmaceutically acceptable carriers. These may be prepared according to methods known to those skilled in the art, for example, as described in U.S. Pat. No. 4,522,811, which is incorporated by reference in its entirety.

Additional pharmaceutical formulations appropriate for administration are known in the art. See Gennaro ed., Remington: The Science and Practice of Pharmacy, 20th ed., Lippincott, Williams & Wilkins (2000); Ansel et al., Pharmaceutical Dosage Forms and Drug Delivery Systems, 7th ed., Lippincott Williams & Wilkins Publishers (1999); Kibbe ed., Handbook of Pharmaceutical Excipients American Pharmaceutical Association, 3rd ed. (2000); and Pharmaceutical Principles of Solid Dosage Forms, Technonic Publishing Co., Inc., Lancaster, Pa., (1993).

The pharmaceutical formulations may be packaged in dosage unit form for ease of administration and uniformity of dosage. “Dosage unit form” as used herein refers to physically discrete units suited as unitary dosages treatment; each unit containing a predetermined quantity of active compound(s) in association with a pharmaceutical carrier or excipient, calculated to produce a desired therapeutic effect.

The invention also provides methods of detecting a vimentin fragment that specifically binds to a human monoclonal antibody produced by the cell line deposited as ATCC accession no. PTA 5411, as well as methods of identifying cells that express a vimentin fragment that specifically binds to a human monoclonal antibody produced by the cell line deposited as ATCC accession no. PTA 5411. In some embodiments, such methods comprise screening a sample or a cell for the presence of a vimentin fragment that specifically binds to a human monoclonal antibody produced by the cell line deposited as ATCC accession no. PTA 5411. In some embodiments, the screening is performed by detecting the presence of the vimentin fragment, or a nucleic acid that encodes the vimentin fragment. The cell may be in vitro or in a subject (e.g, a mammal such as a human).

The invention further provides methods of screening for the presence of a cell proliferative disorder. In some embodiments, methods described comprise analyzing a biological sample for the presence of vimentin fragment that specifically binds to a human monoclonal antibody produced by the cell line deposited as ATCC accession no. PTA 5411. The presence of a vimentin fragment that specifically binds to a human monoclonal antibody produced by the cell line deposited as ATCC accession no. PTA 5411 indicates the presence of a vimentin fragment that specifically binds to a human monoclonal antibody produced by the cell line deposited as ATCC accession no. PTA 5411 in the subject. In some embodiments, the screening is performed by detecting the presence of the vimentin fragment, or a nucleic acid that encodes the vimentin fragment. The cell may be in vitro or in a subject (e.g, a mammal such as a human), located in any tissue or organ. Because the amount of AgRM2 can be measured, the invention further provides methods for detecting levels or amounts of AgRM2.

Cell proliferative disorders screened or identified include benign hyperplasias and tumors, as set forth herein and known in the art. Tumors may be non-metastatic or metastatic; may be in any stage (e.g., a stage I, II, III, IV or V tumor); may be a solid tumor (e.g., sarcoma, carcinoma, melanoma, myeloma, blastoma, glioma, lymphoma or leukemia) or a liquid tumor (e.g., reticuloendothelial or hematopoetic). Specific examples of cell proliferative disorders include cells selected from breast, colon, gut, lung, brain, skin or pancreas.

In some embodiments, examples of cell proliferative disorders include, but not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The invention moreover provides methods of inducing or increasing an immune response to a vimentin fragment that specifically binds to a human monoclonal antibody produced by the cell line deposited as ATCC accession no. PTA 5411. In some embodiments, methods described comparise administering to a subject a sufficient amount of a vimentin fragment that specifically binds to a human monoclonal antibody produced by the cell line deposited as ATCC accession no. PTA 5411 to elicit an immune response to the vimentin fragment in the subject. An immune response may include a cell-mediated or humoral immune response.

The cell may be present in a subject, for example, a mammal (e.g., a human subject) having or at risk of having a cell-proliferative disorder. Thus, the invention also provides methods of treating a cell-proliferative cell disorder in a subject, wherein at least a portion of the cells express AgRM2. In one embodiment, a method includes administering to a subject a sufficient amount of a vimentin fragment that specifically binds to a human monoclonal antibody produced by the cell line deposited as ATCC accession no. PTA 5411 to treat the cell proliferative disorder. Exemplary cell proliferative disorders comprise cells selected from breast, colon, gut, lung, brain, skin or pancreas.

As used herein, the term “proliferate,” and grammatical variations thereof, when used in reference to a cell, tissue or organ, refers to undesirable, excessive or abnormal cell, tissue or organ proliferation, differentiation or survival. Cell proliferative and differentiative disorders include diseases and physiological conditions, both benign and neoplastic, characterized by undesirable, excessive or abnormal cell numbers, cell growth or cell survival in a subject. Specific examples of such disorders include metastatic and non-metastatic tumors and cancers.

Further provided are methods of treating a subject having or at risk of having a tumor. In some embodiments, methods described comprise administering to a subject a sufficient amount of a vimentin fragment that specifically binds to a human monoclonal antibody produced by the cell line deposited as ATCC accession no. PTA 5411 to treat the subject. In some embodiments, methods described comprise administering an anti cell-proliferative or immune system-enhancing treatment or therapy (e.g., an antibody, radioisotope, radiation, a toxic, immunotherapeutic or chemotherapeutic agent, immunotherapy, surgical resection, or hyperthermia), as set forth herein and known to one skilled in the art.

The terms “tumor,” “cancer,” “malignancy,” and “neoplasia” are used interchangeably herein and refer to a cell or population of cells of any cell or tissue origin, whose growth, proliferation or survival is greater than growth, proliferation or survival of a normal counterpart cell, e.g. a cell proliferative or differentiative disorder. Such disorders include, for example, sarcoma, carcinoma, melanoma, myeloma, blastoma, neural (e.g., glioma), and reticuloendothelial or haematopoietic neoplastic disorders (e.g., myeloma, lymphoma or leukemia). Tumors can arise from a multitude of primary tumor types, including but not limited to breast, lung, thyroid, head and neck, brain, lymphoid, gut or gastrointestinal (mouth, esophagus, stomach, small intestine, colon, rectum), genito-urinary tract (uterus, ovary, cervix, bladder, testicle, penis, prostate), kidney, pancreas, liver, bone, muscle, and skin; and may metastasize to secondary sites. Tumors can be non-metastatic or metastatic, and be in any stage, e.g., a stage I, II, III, IV or V tumor; or in remission.

A “solid tumor” refers to neoplasia or metastasis that typically aggregates together and forms a mass. Specific examples include visceral tumors such as melanomas, breast, pancreatic, uterine and ovarian cancers, testicular cancer, including seminomas, gastric or colon cancer, hepatomas, adrenal, renal and bladder carcinomas, lung, head and neck cancers and brain tumors/cancers.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Melanoma refers to malignant tumors of melanocytes and other cells derived from pigment cell origin that may arise in the skin, the eye (including retina), or other regions of the body, including the cells derived from the neural crest that also gives rise to the melanocyte lineage. Additional carcinomas can form from the uterine/cervix, lung, head/neck, colon, pancreas, testes, adrenal gland, kidney, esophagus, stomach, liver and ovary.

Sarcomas refer to malignant tumors of mesenchymal cell origin. Exemplary sarcomas include for example, lymphosarcoma, liposarcoma, osteosarcoma, chondrosarcoma, leiomyosarcoma, rhabdomyosarcoma and fibrosarcoma.

Neural neoplasias include glioma, glioblastoma, meningioma, neuroblastoma, retinoblastoma, astrocytoma, oligodendrocytoma.

A “liquid tumor” refers to neoplasia of the reticuloendothelial or haematopoetic system, such as a lymphoma, myeloma, or leukemia, or a neoplasia that is diffuse in nature. Particular examples of leukemias include acute and chronic lymphoblastic, myeolblastic and multiple myeloma. Typically, such diseases arise from poorly differentiated acute leukemias, e.g., erythroblastic leukemia and acute megakaryoblastic leukemia.

Specific myeloid disorders include, but are not limited to, acute promyeloid leukemia (APML), acute myelogenous leukemia (AML) and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Specific malignant lymphomas include, non-Hodgkin lymphoma and variants, peripheral T cell lymphomas, adult T cell leukemia/lymphoma (ATL), cutaneous T-cell lymphoma (CTCL), large granular lymphocytic leukemia (LGF), Hodgkin's disease, Sezary Syndrome and Reed-Sternberg disease.

Methods of the invention include providing a detectable or measurable improvement in the subject's condition, i.e., providing a therapeutic benefit. A therapeutic benefit is any objective or subjective benefit whether the benefit is transient, temporary, or a longer term improvement in the condition or whether the benefit is a reduction in the severity of an adverse symptom of the condition. Thus, a satisfactory clinical endpoint is achieved when there is an incremental or a partial reduction in the severity, duration or frequency of one or more associated adverse symptoms or complications, or inhibition or reversal of one or more of the physiological, biochemical or cellular manifestations or characteristics of the condition. A therapeutic benefit or improvement (“ameliorate” is used synonymously) therefore need not be destruction of all target cells (e.g., the tumor) or ablation of any particular symptom or complication or all adverse symptoms or complications associated with the disorder. For example, inhibiting an increase in tumor cell mass (stabilization of the tumor) can prolong lifespan and thereby provides a benefit even if only for a few days, weeks or months, even though some or the majority of the tumor remains.

Specific non-limiting examples of therapeutic benefit include a reduction in tumor volume (size or cell mass), inhibiting an increase in tumor volume, slowing or inhibiting tumor progression or metastasis, stimulating tumor cell lysis, necrosis or apoptosis, and reducing tumor metastasis. Examination of a biopsied sample containing a tumor (e.g., blood or tissue sample), can establish whether a reduction in numbers of tumor cells or inhibition of tumor cell proliferation has occurred. Alternatively, for a solid tumor, invasive and non-invasive imaging methods can ascertain a reduction in tumor size, or inhibiting increases in tumor size.

Adverse symptoms and complications associated with tumor, neoplasia, and cancer that can be reduced or decreased include, for example, nausea, lack of appetite, lethargy, pain and discomfort. Thus, a reduction in the severity, duration or frequency of adverse symptoms, an improvement in the subjects subjective feeling, such as increased energy, appetite, and psychological well being, are all examples of therapeutic benefit.

The doses or “sufficient amount” for treatment to achieve a therapeutic benefit or improvement are effective to ameliorate one, several or all adverse symptoms or complications of the condition, to a measurable or detectable extent. Preventing or inhibiting a progression or worsening of the disorder, condition or adverse symptom, is also a satisfactory outcome. Thus, in the case of a cell-proliferative condition or disorder, the amount will be sufficient to provide a therapeutic benefit to the subject or to ameliorate the condition or symptom. The dose may be proportionally increased or reduced as indicated by the status of the disease being treated or a side effect of the treatment.

Doses also considered effective are those that result in reduction of the use of another therapeutic regimen or protocol. For example, a therapeutic benefit is achieved with a vimentin fragment if its administration results in less chemotherapeutic drug, radiation or immunotherapy being required for tumor treatment.

Of course, as is typical for treatment protocols, some subjects will exhibit greater or less response to treatment. For example, appropriate amounts will depend upon the condition treated (e.g., the tumor type or stage of the tumor), the therapeutic effect desired, as well as the individual subject (e.g., the bioavailability within the subject, gender, age, etc.).

The vimentin fragments of the instant invention, and nucleic acids encoding them, may be administered in association with any other therapeutic regimen or treatment protocol. Other treatment protocols include drug treatment (chemotherapy), surgical ressection, hyperthermia, radiotherapy, and immunetherapy, as set forth herein and known in the art. The invention therefore provides methods in which vimentin fragments of the instant invention, and nucleic acids encoding them, are used in combination with any anti-cell proliferative therapeutic regimen or treatment protocol, such as those set forth herein or known in the art.

Radiotherapy includes internal or external delivery to a subject. For example, alpha, beta, gamma and X-rays may be administered to the subject externally without the subject internalizing or otherwise physically contacting a radioisotope. Specific examples of X-ray dosages administered range from daily doses of 50 to 200 roentgens for prolonged periods of time (3 to 5/week), to single doses of 2000 to 6000 roentgens. Dosages vary widely, and depend on duration of exposure, the half-life of the isotope, the type of radiation emitted, the cell type and location treated and the progressive stage of the disease.

The term “subject” or “patient” refers to an animal, typically a mammalian animal, such as a non-human primate (gorillas, chimpanzees, orangutans, macaques, gibbons), a domestic animal (dogs and cats), a farm animal (horses, cows, goats, sheep, pigs), an experimental animal (mouse, rat, rabbit, guinea pig) and a human. Subjects include disease model animals (e.g., such as mice and non-human primates) for testing in vivo efficacy of the vimentin fragments of the instant invention and nucleic acids encoding vimentin fragments of the invention (e.g., a tumor animal model). Human subjects include adults, and children, for example, newborns and older children, between the ages of 1 and 5, 5 and 10 and 10 and 18 years.

Subjects and/or patients include humans having or at risk of having a cell-proliferative disorder, such as subjects having a cell or tissue that expresses AgRM2, or subjects that have a family history of, are genetically predisposed to, or have been previously afflicted with a cell proliferative disorder. Thus, subjects at risk for developing cancer can be identified with genetic screens for tumor associated genes, gene deletions or gene mutations. Subjects at risk for developing breast cancer lack Brca1, for example. Subjects at risk for developing colon cancer have deleted or mutated tumor suppressor genes, such as adenomatous polyposis coli (APC), for example. Subjects include candidates for anti-tumor or immune system-enhancing therapy, or those that are undergoing or have undergone an anti cell-proliferative or immune system-enhancing therapy (e.g., subjects in remission).

The term “patient” also refers to a living subject who has presented at a clinical setting with a particular symptom or symptoms suggesting the need for treatment with a therapeutic agent of the invention. The treatment may either be generally accepted in the medical community or it may be experimental. In some embodiments, the patient is a mammal, including animals such as dogs, cats, pigs, cows, sheep, goats, horses, rats, and mice. In some embodiments, the patient is a human. A patient's diagnosis can alter during the course of disease progression, either spontaneously or during the course of a therapeutic regimen or treatment.

Further provided are methods of screening for or identifying an inhibitor or stimulator of expression of a vimentin fragment that specifically binds to a human monoclonal antibody produced by the cell line deposited as ATCC accession no. PTA 5411. In one embodiment, a method includes contacting a cell that expresses or is capable of expressing a vimentin fragment that specifically binds to a human monoclonal antibody produced by the cell line deposited as ATCC accession no. PTA 5411 with a test compound; and detecting expression of said vimentin fragment that specifically binds to a human monoclonal antibody produced by the cell line deposited as ATCC accession no. PTA 5411. A change in expression indicates that the test compound is an inhibitor or stimulator of expression of a vimentin fragment that specifically binds to a human monoclonal antibody produced by the cell line deposited as ATCC accession no. PTA 5411. In various aspects, the contacting is in solution, in solid phase, in vivo or in vitro.

Additionally provided are methods of screening for or identifying a subject having or at risk of having a cell proliferative disorder (e.g., a cell proliferative disorder in a tissue selected from breast, colon, gut, lung, brain, skin or pancreas). In one embodiment, a method includes analyzing for the expression of a vimentin fragment that specifically binds to a human monoclonal antibody produced by the cell line deposited as ATCC accession no. PTA 5411. The presence of vimentin polypeptide sequence that specifically binds to a human monoclonal antibody produced by the cell line deposited as ATCC accession no. PTA 5411 in the tissue identifies the subject as having or at risk of having a cell proliferative disorder.

Methods that can be used in the instant invention in the fields of molecular genetics and genetic engineering are described in “Molecular Cloning: A Laboratory Manual” 2nd Ed. (Sambrook et al., 1989); “Oligonucleotide Synthesis” (M. J. Gait, ed., 1984); “Animal Cell Culture” (R. I. Freshney, ed., 1987); the series “Methods in Enzymology” (Academic Press, Inc.); “Gene Transfer Vectors for Mammalian Cells” (J. M. Miller & M. P. Calos, eds., 1987); “Current Protocols in Molecular Biology” and “Short Protocols in Molecular Biology, 3rd Edition” (F. M. Ausubel et al., eds., 1987 & 1995); and “Recombinant DNA Methodology II” (R. Wu ed., Academic Press 1995).

Methods in immunology that can be used in the instant invention include raising, purifying and modifying antibodies; and the design and execution of immunoassays such as immunohistochemistry can be found in the Handbook of Experimental Immunology (D. M. Weir & C. C. Blackwell, eds.); Current Protocols in Immunology (J. E. Coligan et al., eds., 1991); and R. Masseyeff, W. H. Albert, and N. A. Staines, eds., Methods of Immunological Analysis (Weinheim: VCH Verlags GmbH, 1993).

Methods in cell culture and media collection that can be used in the instant invention can be found in Large Scale Mammalian Cell Culture (Hu et al., Curr. Opin. Biotechnol. 8: 148 (1997); Serum-free Media (K. Kitano, Biotechnology 17:7 3 (1991); Large Scale Mammalian Cell Culture (Curr. Opin. Biotechnol. 2: 375 (1991); and Suspension Culture of Mammalian Cells (Birch et al., Bioprocess Technol. 19: 251 (1990), which are all incorporated by reference in their entireties.

Unless otherwise defined, all technical and scientific terms used herein have the same meaning as commonly understood by one of ordinary skill in the art to which this invention relates. Although methods and materials similar or equivalent to those described herein may be used in the practice or testing of the invention, suitable methods and materials are described herein.

All publications, patents and other references cited herein are incorporated by reference in their entirety, including any tables, drawings or figures. In case of conflict, the present specification, including definitions, will control.

As used herein, singular forms “a”, “and,” and “the” include plural referents unless the context clearly indicates otherwise. Thus, for example, reference to “a vimentin polypeptide” includes a plurality of vimentin polypeptides, and reference to “a sequence” may include reference to all or a potion of or one or more sequences, and so forth.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

The RM2 antibody is used to identify the AgRM2 antigen in a Western Blot. The polypeptide band was excised and sequenced. The polypeptide sequence was found to be a truncated version of vimentin (FIG. 1). The beginning amino terminal residue of AgRM2 was identified as residue #29 of normal vimentin. The gene sequence was identified from mRNA isolated from the SK-MEL-28 cell line (available from the ATCC) and sequenced by standard methods. The sequence is illustrated in FIG. 1.

### Example 2

The sequence of the isolated polypeptide is also determined by gel isolation, excision, and sequenced. The beginning terminal residue is residue #28, #30, #31, #32, #33, #34, #35, #36, #37, #38, #39, or #40 of normal vimentin.

### Example 3

The RM2 antibody is used to identify the AgRM2 antigen in a Western Blot prepared from Sezary Syndrome cells (a cutaneous T-cell lymphoma). The polypeptide band is excised and sequenced. The polypeptide sequence is found to be a truncated version of vimentin.

We claim:

1. An isolated polypeptide that specifically binds to a human monoclonal antibody produced by a cell line deposited as ATCC accession no. PTA 5411.

2. The polypeptide of claim 1, wherein said polypeptide's amino acid backbone's sequence comprises an amino acid sequence that lacks at least about 20 amino acids from said polypeptide's amino terminus relative to said sequence set forth in SEQ ID NO:1.

3. The polypeptide of claim 1, wherein said polypeptide's amino acid backbone's sequence comprises an amino acid sequence that lacks at least about 30 amino acids from said polypeptide's amino terminus relative to said amino acid sequence set forth in SEQ ID NO:1.

4. The polypeptide of claim 1, wherein said polypeptide's amino acid backbone's sequence comprises an amino acid sequence that lacks at least about 40 amino acids from said polypeptide's amino terminus relative to said sequence set forth in SEQ ID NO:1.

5. The polypeptide of claim 1, wherein said polypeptide is less than about 400 amino acids in length.

6. The polypeptide of claim 1, wherein said polypeptide is a vimentin variant.

7. An isolated polypeptide that comprises a fragment of an amino acid sequence as set forth in SEQ ID NO:1 wherein said fragment is a carboxy-terminal fragment that specifically binds to a human monoclonal antibody produced by a cell line deposited as ATCC accession no. PTA 5411, and said polypeptide's amino acid sequence comprises less than about 440 amino acids of said sequence set forth in SEQ ID NO:1.

8. The isolated polypeptide of claim 7, wherein said polypeptide's amino acid sequence comprises less than about 400 amino acids of said amino acid sequence set forth in SEQ ID NO:1.

9. A nucleic acid encoding said polypeptide of claim 1.

10. A transformed cell that contains said polypeptide of claim 1.

11. A vector comprising a nucleic acid encoding said polypeptide of claim 1.

12. A transformed cell comprising a nucleic acid encoding said polypeptide of claim 1.

13. A pharmaceutical composition comprising said polypeptide of claim 1, and a pharmaceutically acceptable carrier.

14. A kit comprising said polypeptide of claim 1.

15. A method of identifying a cell that expresses a polypeptide that specifically binds to a human monoclonal antibody produced by a cell line deposited as ATCC accession no. PTA 5411, comprising screening said cell for expression of said polypeptide.

16. The method of claim 15, wherein said screening comprises detecting a nucleic acid that encodes said polypeptide.

17. A method of screening for a cell proliferative disorder comprising analyzing a biological sample for a polypeptide that specifically binds to a human monoclonal antibody produced by a cell line deposited as ATCC accession no. PTA 5411.

18. The method of claim 17, wherein said screening comprises detecting said polypeptide's presence.

19. The method of claim 17, wherein said screening comprises detecting a nucleic acid that encodes said polypeptide.

20. The method of claim 17, wherein said cell proliferative disorder is a tumor.

21. The method of claim 17, wherein said cell proliferative disorder is derived from cells selected from the group consisting of breast, colon, gut, lung, brain, skin and pancreas cells.

22. A method of inducing or increasing an immune response to a polypeptide that specifically binds to a human monoclonal antibody produced by a cell line deposited as ATCC accession no. PTA 5411 comprising administering to a subject a sufficient amount of said polypeptide to elicit said immune response to said polypeptide in said subject.

23. The method of claim 22, wherein said immune response comprises a cell-mediated immune response.

24. The method of claim 22, wherein said immune response comprises a humoral immune response.

25. A method of treating a cell proliferative disorder comprising administering to a subject a sufficient amount of a polypeptide that specifically binds to a human monoclonal antibody produced by a cell line deposited as ATCC accession no. PTA 5411 to treat said cell proliferative disorder.

26. The method of claim 25, wherein said cell proliferative disorder is a tumor.

27. A method of treating a subject having, or at risk of having, a tumor comprising administering to said subject a sufficient amount of a polypeptide that specifically binds to a human monoclonal antibody produced by a cell line deposited as ATCC accession no. PTA 5411 to treat said subject.

28. The method of claim 27 further comprising administering an additional anti-tumor or immune system-enhancing agent or treatment.

29. The method of claim 28, wherein said additional anti-tumor or immune system-enhancing agent is selected from the group consisting of an antibody, a radioisotope, a toxic agent, an immunotherapeutic agent, a chemotherapeutic agent, external radiation, and any combination thereof.

30. A method of identifying an inhibitor or stimulator of the expression of a polypeptide that specifically binds to a human monoclonal antibody produced by a cell line deposited as ATCC accession no. PTA 5411 comprising:
a) contacting a cell that is capable of expressing said polypeptide that specifically binds to a human monoclonal antibody produced by said cell line with a test compound; and
b) detecting expression of said polypeptide,
wherein a change said expression of said polypeptide indicates that the test compound is an inhibitor or stimulator of said expression of said polypeptide.

31. A method of screening a subject having or at risk of having a cell proliferative disorder, comprising the steps of:
analyzing said subject for expression of a polypeptide that specifically binds to a human monoclonal antibody produced by a cell line deposited as ATCC accession no. PTA 5411,
wherein said polypeptide's presence in the tissue identifies said subject as having or at risk of having a cell proliferative disorder.

32. The method of claim 31, wherein said cell proliferative disorder is derived from a tissue selected from breast, colon, gut, lung, brain, skin and pancreas.
